(19) United States
(12) Patent Application Publication
(10) Pub. No.: US 2012/0294803 A1
(43) Pub. Date: Nov. 22, 2012

(54) CXCR4 ANTAGONISTS FOR IMAGING OF CANCER AND INFLAMMATORY DISORDERS

(75) Inventors: Hyunsuk Shim, Atlanta, GA (US); Dennis C. Liotta, Atlanta, GA (US); Mark M. Goodman, Atlanta, GA (US); Aizhi Zhu, Stone Mountain, GA (US)

(73) Assignee: EMORY UNIVERSITY, Atlanta, GA (US)

(21) Appl. No.: 13/574,273

(22) PCT Filed: Jan. 27, 2011
(86) PCT No.: PCT/US2011/022696

§ 371 Date: Aug. 7, 2012

Related U.S. Application Data
(60) Provisional application No. 61/298,674, filed on Jan. 27, 2010.

(51) Int. Cl. A61K 51/04; A61P 29/00; A61P 35/04; C07D 413/14; A61P 35/00

(52) U.S. Cl. 424/1.89; 544/122

(57) ABSTRACT

The invention provides radiolabeled CXCR4 antagonists, compositions and methods of use for imaging of chemokine CXCR4 receptors and biological conditions associated with the expression of CXCR4 receptors, including cancer, metastasis, and inflammatory disorders.

### CROSS-REFERENCE TO RELATED APPLICATIONS

This application claims priority to U.S. Provisional Application No. 61/298,674 filed Jan. 27, 2010.

### FIELD

The invention relates to CXCR4 receptor antagonists for use as imaging agents and methods for imaging of CXCR4 chemokine receptors. The compounds and methods are useful in the imaging and diagnosis of various conditions, including cancer, metastasis and inflammatory disorders.

### BACKGROUND

Cancer is currently the second leading cause of death in developed nations. In 2004, the American Cancer Society estimated that approximately 1.37 million new cases were diagnosed in the U.S. alone, and approximately 550,000 deaths occurred due to cancer (American Cancer Society, Cancer Facts & Figures 2004, see URL: http://www.cancer.org/docroot/STT/stt_{—}0.asp). Metastasis, the spread and growth of tumor cells to distant organs, is the most devastating attribute of cancer. Most morbidity and mortality associated with certain types of cancer, such as breast cancer, is associated with disease caused by metastatic cells rather than by the primary tumor. Therapy for metastasis currently relies on a combination of early diagnosis and aggressive treatment of the primary tumor.

The establishment and growth of metastases at distant sites is thought to depend on interactions between tumor cells and the host environment. Metastasis is the result of several sequential steps and represents a highly organized, non-random and organ-selective process. Although a number of mediators have been implicated in the metastasis of breast cancer, the precise mechanisms determining the directional migration and invasion of tumor cells into specific organs remain to be established. An incomplete understanding of the molecular and cellular mechanisms underlying metastasis has hindered the development of effective therapies that would eliminate or ameliorate this condition.

Chemokines are a superfamily of small cytokines that induce, through their interaction with G-protein-coupled receptors, cytoskeletal rearrangements and directional migration of several cell types. These secreted proteins act in a coordinated fashion with cell-surface proteins to direct the homing of various subsets of cells to specific anatomical sites (Morales, et al. (1999) Proc Natl Acad Sci USA 96: 14470-14475; Homey, B., et al. (2000) J Immunol 164: 3465-3470; Peled, et al. (1999) Science 283: 845-848; Forster, et al. (1999) Cell 99: 23-33).

Chemokines are considered to be principal mediators in the initiation and maintenance of inflammation. They have also been found to play an important role in the regulation of endothelial cell function, including proliferation, migration and differentiation during angiogenesis and re-endothelialization after injury (Gupta et al. (1998) J Biol Chem, 7:4282-4287). CXCL12 (Stromal cell derived factor 1 (SDF-1)) is a chemokine that interacts specifically with CXCR4. When CXCL12 binds to CXCR4, CXCR4 activates Gαᵢ-protein-mediated signaling (pertussis toxin-sensitive), including downstream kinase pathways such as Ras/MAP Kinases and phosphatidylinositol 3-kinase (PI3K)/Akt in lymphocyte, megakaryocytes, and hematopoietic stem cells (Bleul, et al. (1996) Nature 382: 829-833; Deng, et al. (1997) Nature 388: 296-300; Kijowski, et al. (2001) Stem Cells 19: 453-466; Majka, et al. (2001) Folia. Histochem. Cytobiol. 39: 235-244; Sotsios, et al. (1999) J. Immunol. 163: 5954-5963; Vlahakis, et al. (2002) J. Immunol. 169: 5546-5554). In mice transplanted with human lymph nodes, CXCL12 induces CXCR4-positive cell migration into the transplanted lymph node (Blades et al. (2002) J. Immunol. 168: 4308-4317). These results imply that the interaction between CXCL12 and CXCR4 directs cells to the organ sites with high levels of CXCL12.

Recently, studies have shown that CXCR4 interactions may regulate the migration of metastatic cells. Hypoxia, a reduction in partial oxygen pressure, is a microenvironmental change that occurs in most solid tumors and is a major inducer of tumor angiogenesis and therapeutic resistance. Hypoxia increases CXCR4 levels (Staller, et al. (2003) Nature 425: 307-311). Microarray analysis on a sub-population of cells from a bone metastatic model with elevated metastatic activity showed that one of the genes increased in the metastatic phenotype was CXCR4. Furthermore, overexpression CXCR4 in isolated cells significantly increased the metastatic activity (Kang, et al. (2003) Cancer Cell 3: 537-549). In samples collected from various breast cancer patients, Muller et al. (Muller, et al. (2001) Nature 410: 50-56) found that CXCR4 expression level is higher in primary tumors relative to normal mammary gland or epithelial cells. These results suggest that the expression of CXCR4 on cancer cell surfaces may direct the cancer cells to sites that express high levels of CXCL12. Consistent with this hypothesis, CXCL12 is highly expressed in the most common destinations of breast cancer metastasis including lymph nodes, lung, liver, and bone marrow. Moreover, CXCR4 antibody treatment has been shown to inhibit metastasis to regional lymph nodes when compared to control isotypes that all metastasized to lymph nodes and lungs (Muller, et al. (2001) Nature 410: 50-56).

In addition to regulating migration of cancer cells, CXCR4-CXCL12 interactions may regulate vascularization necessary for metastasis. Blocking either CXCR4/CXCL12 interaction or the major G-protein of CXCR4/CXCL12 signaling pathway (Gαᵢ) inhibits VEGF-dependent neovascularization. These results indicate that CXCL12/CXCR4 controls VEGF signaling systems that are regulators of endothelial cell morphogenesis and angiogenesis. Numerous studies have shown that VEGF and MMPs actively contribute to cancer progression and metastasis.

Several groups have identified chemokines including CXCR4 as a target for treatment of metastatic cancers. For example, PCT Publication Nos. WO 01/38352 to Schering Corporation, WO 04/059285 to Protein Design Labs, Inc., and WO 04/024178 to Burger generally describe methods of treating diseases and specifically inhibiting metastasis by blocking chemokine receptor signaling.

PCT Publication No. WO 2008/008854, filed Jul. 11, 2007 describes certain compounds for the treatment of medical disorders mediated by CXCR4. These compounds include two nitrogen linked cyclic substituents off a central aromatic or cyclic alkyl or heteroalkyl.

Oncology therapy has generally relied on tumor-targeting cytotoxic therapy or tumor imaging, rather than molecular-target therapy or imaging of molecular targets to enhance early detection of cancer.

Positron Emission Tomography (PET) is am imaging technique that produces an image of the body. The technique involves detection of pairs of gamma rays emitted indirectly by a positron-emitting radionuclide (tracer), which is introduced into the body on a biologically active molecule. Images of tracer concentration in the body are then reconstructed by computer analysis.

PET is a molecular target-specific imaging technology and is fundamentally different from magnetic resonance imaging (MRI) and computed tomography (CT). MRI and CT are useful for clinical diagnosis when the disease process causes significant anatomic alterations. In oncology, PET is particularly useful for differentiating tumors from post-surgical changes or radiation necrosis, distinguishing benign from malignant lesions, identifying the optimal site for biopsy, staging cancers, and monitoring the response to therapy. (Van den Abbeele, et al. (2002) Eur. J. Cancer 38 Suppl. 5: S60-5; Langleben, et al. (2000) J. Nucl. Med. 41(11):1861-7; and Dobos, et al. (2002) Hematol. Oncol. Clin. North Am. 16(4): 875-85.) Ultimately, this leads to decreased morbidity and mortality as well as a reduction in medical resource requirement. PET is a powerful, proven diagnostic imaging modality that displays information unobtainable through any other means. However, current PET imaging agents used in clinical oncology either lack specificity for the cancer, are not accurate predictors of metastasis, or are eliminated too quickly or too slowly from the body for optimal imaging.

Fluorine-18 is an attractive positron-emitting nuclide for use in PET because its relatively long 109-minute half-life is sufficient for the study of drug delivery and distribution, and is conducive to distribution through regional radiopharmacies for widespread clinical usage. A glucose analog, [18F]deoxyglucose (FDG), the most common radiotracer for cancer imaging, is limited in specificity because it only reports cells with high glucose uptake. The use of FDG can also be problematic in the head and neck region where physiologic and iatrogenic post-treatment changes can markedly confound image interpretation (Blodgett et al. (2005) Radiographics 25(4); 897-912).

U.S. Patent Application Publication No. 2007/0258893 describes imaging compositions and methods for detection of biological conditions associated with expressin of CXCR4 receptors comprising certain radiolabeled CXCR4 peptide antagonists.

AMD3100 (1,1′-[1,4-Phenylenebis(methylene)]bis [1,4,8,11-tetraazacyclotetradecane]octohydrobromide dehydrate; also known as Plerixafor) is a known CXCR4 antagonist that has been approved for the mobilization of hematopoietic stem cells by the U.S. Food and Drug Administration. While it has been established that AMD3100 is an antagonist of CXCR4 in vitro, it also appears to have more activities than simple CXCR4 antagonism in vivo. Recently, Jacobsen, et al. labeled AMD3100 with the radioisotope ⁶⁴Cu to produce ⁶⁴Cu-AMD3100 (Jacobsen, et al. (2009) Bioorg. Med. Chem. 17(4): 1486-93). Biodistribution of ⁶⁴Cu-AMD3100 showed accumulation in CXCR4-expressing organs and a binding affinity to CXCR4 of 62.7 μM.

There remains a need for imaging agents that can provide sensitive and rapid detection of pathological conditions associated with the expression of CXCR4 receptors, in particular of cancer and cancer metastasis.

It is therefore an object of the invention to provide new imaging agents, methods and compositions for detection of the expression of CXCR4 receptors.

It is a more specific object of the invention to provide new imaging agents, methods and compositions for the detection and diagnosis of cancer or cancer metastases.

In certain embodiments, the disclosure relates to methods of producing N⁴-(442-18-fluoro,5-fluoropyrimidin-4-ylamino)methyl)benzyl)-N²-(2-morpholino ethyl)pyrimidine-2,4-diamine comprising mixing N⁴-(4-((2-halogen,5-fluoropyrimidin-4-ylamino)methyl)benzyl)-N²-(2-morpholino ethyl)pyrimidine-2,4-diamine and a composition comprising ¹⁸F under conditions such that N⁴-(4-((2-¹⁸fluoro,5-fluoropyrimidin-4-ylamino)methyl)benzyl)-N2-(2-morpholinoethyl)pyrimidine-2,4-diamine is formed.

### SUMMARY

Compounds, compositions and methods for imaging the expression of CXCR4 receptors for detection of pathological conditions or diseases associated with CXCR4 receptor expression, including but not limited to cancer, metastasis, tumors, angiogenesis and inflammation. In particular aspects, imaging compositions and methods for the detection, quantification, or identification of cancer cells and/or cancer cell metastases are provided. In certain embodiments, the imaging compositions comprise labeled CXCR4 antagonists which may be used to image CXCR4 expression by PET, SPECT, MRS, MRI and optical imaging. The labels of the labeled CXCR4 antagonist can include isotopes suitable for PET and SPECT imaging, for example radioisotopes including ¹⁸F, ⁷⁶Br, ¹²³I, ¹²⁴I, ¹²⁵I, or ¹³¹I; I; isotopes suitable for magnetic resonance imaging techniques, for example ¹¹C or ¹³C; and labels suitable for optical imaging, for example dyes.

In particular embodiments, the labeled CXCR4 antagonist is a radiolabeled CXCR4 antagonist. Radiolabeled CXCR4 antagonists may used to detect pathological conditions or diseases associated with CXCR4 receptor expression by PET or SPECT imaging. The radiolabeled CXCR4 antagonists include, for example, compounds of Formula I, or a pharmaceutically acceptable salt, ester or prodrug thereof:

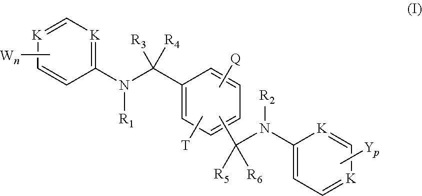

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl, aralkyl, aryl, heteroarylalkyl and heterocyclylalkyl;
wherein at least one of Q, T, W and Y is a radioisotope selected from the group consisting of ¹⁸F, ⁷⁶Br, ¹²³I, ¹²⁴I, ¹²⁵I, or ¹³¹I;
n is 0, 1, 2 or 3;
p is 0, 1, 2 or 3;
R¹, R², R³, R⁴, R⁵ and R⁶ are independently selected from H, straight chain, branched or cyclic alkyl, alkenyl, alkynyl, aralkyl, aryl heteroaryl, acyl (RC—) and imidoyl (RC(NH)— or RC(NR′)—) groups.

In particular embodiments, at least one of Q, T, W and Y is ¹⁸F.

In one embodiment, the radiolabeled CXCR4 antagonist is a compound of formula I-1, or a pharmaceutically acceptable salt, ester or prodrug thereof, is provided:

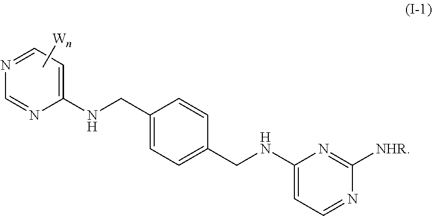

In another embodiment, the radiolabeled CXCR4 antagonist is a compound of formula I-2, or a pharmaceutically acceptable salt, ester or prodrug thereof, is provided:

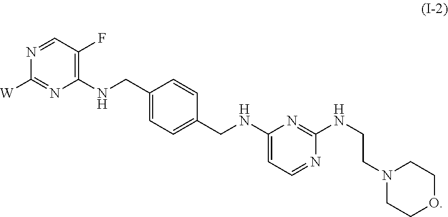

In a particular embodiment, the radiolabeled CXCR4 antagonist is of the structure:

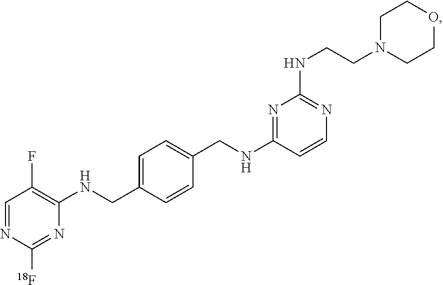

which is referred to herein as [¹⁸F]M508F.

The CXCR4 antagonist interferes with ligand binding to a CXCR4 receptor or homologue thereof; in particular, the CXCR4 antagonist prevents the CXCR4 receptor from binding the ligand CXCL12. In one embodiment the CXCR4 antagonist comprises a radiolabel that is detectable by PET or SPECT imaging. In one embodiment, the radiolabel is a positron-emitting nuclide suitable for use in PET or SPECT imaging, for example ¹⁸F, ⁷⁶Br, ¹²³I, ¹²⁴I, ¹²⁵ or ¹³¹I. In certain embodiments, the radiolabel is suitable for PET imaging, for example ¹⁸F or ⁷⁶Br. In certain embodiments, the radiolabel is suitable for SPECT imaging, for example ¹²³I, ¹²⁴I, ¹²⁵I, or ¹³¹I. In a particular subembodiment, the radiolabel is ¹⁸F.

In one embodiment, the labeled CXCR4 antagonist is labeled with ¹¹C or ¹³C and is suitable for MRI or MRS imaging.

In one embodiment, the labeled CXCR4 antagonist is labeled with a dye that is suitable for optical imaging, for example a near-infrared dye.

In one embodiment, an imaging composition comprising a labeled CXCR4 antagonist and a pharmaceutically acceptable carrier is provided.

In another embodiment, a method of imaging comprising providing an imaging probe including radiolabeled CXCR4 antagonist, administering to the specimen to be imaged with an effective amount of the imaging probe, and making an image, for example a radiographic image. In particular, the methods include imaging expression of CXCR4 receptors, where the expression of CXCR4 receptors is associated with one or more conditions selected from: inflammation, cancer, a tumor, angiogenesis, and metastasis.

In other embodiments, methods of imaging a condition associated with expression of CXCR4 receptors in a host are provided. Such methods include administering to the host a detectably effective amount of a composition including a radiolabeled CXCR4 antagonist, and creating an image of the location and distribution of the labeled CXCR4 antagonist in the host with an imaging apparatus. The labeled CXCR4 antagonist binds to CXCR4 receptors, and the intensity of uptake of labeled CXCR4 antagonist is related to the expression level of CXCR4 receptors in the host, where the expression level of CXCR4 receptors is associated with one or more disorders.

In another aspect, methods of predicting metastasis of a tumor and/or cancer are provided. An exemplary embodiment of a method of predicting metastasis includes contacting a specimen having tumor cells with an amount effective for detection of a composition of radiolabeled CXCR4 antagonist and creating a radiographic image of the location and distribution of the radiolabeled CXCR4 antagonist in the tumor cells with an imaging apparatus. In such methods, the radiolabeled CXCR4 antagonist, for example [¹⁸F]M508F, binds to CXCR4 receptors, and the intensity of uptake of radiolabeled CXCR4 antagonist, by the tumor cells is related to the metastatic potential of the tumor cells.

In another embodiment, methods of determining the effectiveness of a drug on a condition associated with expression of CXCR4 receptors, such as, but not limited to, cancer, cancer metastasis, diseases of vasculature, inflammatory and degenerative diseases. In exemplary embodiments, such methods include administering an amount of the drug to a host with cancer, administering a detectably effective amount of a composition of radiolabeled CXCR4 antagonist, to a host, creating a radiographic image of the location and distribution of the radiolabeled CXCR4 antagonist, in the host with an imaging apparatus, and determining an amount of radiolabeled CXCR4 antagonist taken up by host cancer cells. The amount of uptake of radiolabeled CXCR4 antagonist by the host is related to the effectiveness of the drug.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1a is a graph of the result of a competition binding assay using metastatic 686LN cells incubated with [¹⁸F]M508F and increasing concentrations of nonradioactive [¹⁹F]M508F. FIG. 1b is a graph of the result of a competition binding assay using metastatic 686LN cells incubated with [¹⁸F]M508F and increasing concentrations of CXCL12 (ligand for CXCR4).

FIG. 2 is a graph of the biodistribution of [¹⁸F]M508F in normal C57BI/6 mice at 15, 30, 60 and 120 minutes post-injection.

FIG. 3 shows a sagittal image of a mouse bearing CXCR4-positive SCCHN tumors in its neck. The image was obtained 30 minutes post-injection of [¹⁸F]M508F.

FIG. 4 shows PET/CT scan images of the feet and tail of control and edema-afflicted mice. The images were obtained 30 minutes post-injection of [¹⁸F]M508F.

FIG. 5a shows PET/CT scan images of the lungs of control and mouse with lung metastases. These images were obtained 60 minutes post-injection of [¹⁸F]M508F. FIG. 5b shows PET/CT scan images of the lungs of control and mouse with lung inflammation due to 20 Gy-irradiation. The images were obtained 60 minutes post-injection of [¹⁸F]M508F.

### DETAILED DESCRIPTION OF THE INVENTION

Imaging agents, compositions and methods for imaging the expression, and particularly the overexpression, of certain surface cell receptors that are indicators of a disease or condition are provided. In particular, the present disclosure relates to compositions and methods for imaging the expression of CXCR4 chemokine receptors for imaging conditions/diseases associated with CXCR4 receptor expression, including, but not limited, to inflammation, cancer, angiogenesis, tumors, and metastasis. Embodiments of the present disclosure include compositions and methods for the detection and staging of cancer and/or tumors and the prediction and/or diagnosis of metastasis. In certain embodiments, the present disclosure provides compositions and methods for imaging CXCR4 mediated pathology (e.g. cancer, angiogenesis, inflammation, and metastasis) by administering a labeled CXCR4 antagonist to a host in an effective amount, for example in an amount sufficient to detect a cell expressing a CXCR4 receptor or homologue thereof. The imaging methods can include gamma imaging, for example PET or SPECT, magnetic reseonance imaging (MRI) or magnetic resonance spectroscopy (MRS), and optical imaging.

In particular embodiments, the labeled CXCR4 antagonist is a radiolabeled CXCR4 antagonist, for example a compound of formula I-V as described herein. Another embodiment provides uses of a radiolabeled CXCR4 antagonist for the manufacture of an imaging composition for the imaging and staging of CXCR4 mediated pathologies including, but not limited to, cancer and tumor metastasis. Still another embodiment provides uses of a radiolabeled CXCR4 antagonist for an imaging composition for the detection and prediction of tumor cell metastasis in a mammal.

In certain embodiments, the CXCR4 antagonists described herein are labeled with a radiolabel suitable for imaging with gamma, PET or SPECT imaging technology, preferably an isotope suitable for PET imaging. In other embodiments, the CXCR4 antagonists described herein are labeled with ¹¹C or ¹³C, for example by incorporating into the carbons of the CXCR4 antagonist, for MRI or MRS imaging. In other embodiments, the CXCR4 antagonists described herein are labeled with a dye, for example a near-infrared dye, suitable for optical imaging. Exemplary compositions described here can be used to image, detect, and/or predict cancer, in particular the spread of cancer, within an organism.

### DEFINITIONS

Unless defined otherwise, all technical and scientific terms used herein have the same meaning as commonly understood by one of ordinary skill in the art to which this disclosure belongs.

As will be apparent to those of skill in the art, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Unless indicated otherwise, parts are parts by weight, temperature is in ° C., and pressure is at or near atmospheric. Standard temperature and pressure are defined as 20° C. and 1 atmosphere. Embodiments of the present disclosure will employ, unless otherwise indicated, techniques of medicine, pharmacology, nuclear chemistry, biochemistry, molecular biology, and the like, which are within the skill of the art. Such techniques are explained fully in the literature.

The term “CXCR4 antagonist” refers to a compound or substance that interferes or inhibits the biological activity of the CXCR4 receptor including, but not limited to, the binding of a ligand to the receptor.

As used herein, the term “imaging probe”, “imaging agent”, or “imaging compound” refers to the labeled compounds of the present disclosure that are capable of serving as imaging agents and whose uptake is related to the expression level of certain surface cell receptors, particularly CXCR4 receptors.

A “pharmaceutically acceptable carrier” refers to a biocompatible solution, having due regard to sterility, pH, isotonicity, stability, and the like and can include any and all solvents, diluents (including sterile saline, Sodium Chloride Injection, Ringer's Injection, Dextrose Injection, Dextrose and Sodium Chloride Injection, Lactated Ringer's Injection and other aqueous buffer solutions), dispersion media, coatings, antibacterial and antifungal agents, isotonic agents, and the like. The pharmaceutically acceptable carrier may also contain stabilizers, preservatives, antioxidants, or other additives, which are well known to one of skill in the art, or other vehicle as known in the art.

As used herein, “pharmaceutically acceptable salts” refer to derivatives of the disclosed compounds wherein the parent compound is modified by making non-toxic acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

The pharmaceutically acceptable salts of the present disclosure can be synthesized from a parent compound that contains a basic or acidic moiety by conventional chemical methods. Generally, such salts can be prepared by reacting free acid forms of these compounds with a stoichiometric amount of the appropriate base (e.g., Na, Ca, Mg, or K, hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., (1985).

By “administration” is meant introducing a compound of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

As used herein, the term “inhibit” and/or “reduce” generally refers to the act of reducing, either directly or indirectly, a function, activity, or behavior relative to the natural, expected, or average or relative to current conditions.

As used herein, the term “host”, “organism”, “individual” or “subject” includes humans, mammals (e.g., cats, dogs, horses, etc.), living cells, and other living organisms. A living organism can be as simple as, for example, a single eukaryotic cell or as complex as a mammal. “Patient” refers to an individual or subject who has undergone, is undergoing, or will undergo treatment.

In accordance with the present disclosure, “an effective amount” or “a detectably effective amount” of the imaging agent of the present disclosure is defined as an amount sufficient to yield an acceptable image using equipment that is available for clinical use. A detectably effective amount of the imaging agent of the present disclosure may be administered in more than one injection. The detectably effective amount of the imaging agent of the present disclosure can vary according to factors such as the degree of susceptibility of the individual, the age, sex, and weight of the individual, idiosyncratic responses of the individual, the dosimetry, and the like. Detectably effective amounts of the imaging agent of the present disclosure can also vary according to instrument and film-related factors. Optimization of such factors is well within the level of skill in the art.

The term “therapeutically effective amount” as used herein refers to that amount of the compound being administered which will relieve to some extent one or more of the symptoms of the disorder being treated. In reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer, or angiogenesis.

“Cancer”, “tumor”, and “precancerous” as used herein, shall be given their ordinary meaning, as general terms for diseases in which abnormal cells divide without control. Cancer cells can invade nearby tissues and can spread through the bloodstream and lymphatic system to other parts of the body. Various forms of cancer are discussed in greater detail below. It should be noted that cancerous cells, cancer, and tumors are sometimes used interchangeably in the disclosure.

The term “alkyl”, as used herein, unless otherwise specified, refers to a saturated straight, branched, or cyclic, primary, secondary, or tertiary hydrocarbon of typically C₁ to C₁₀, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term optionally includes substituted alkyl groups. Moieties with which the alkyl group can be substituted are selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

Whenever any range is specified in the application, this range includes independently each and every element of the range. In one, non-limiting example, when the terms “C₁-C₅ alkyl”, “C₂-C₅ alkenyl”, “C₁-C₅ alkoxy”, “C₂-C₅ alkenoxy”, “C₂-C₅ alkynyl”, and “C₂-C₅ alkynoxy” are used, these are considered to include, independently, each member of the group, such that, for example, C₁-C₅ alkyl includes straight, branched and where appropriate cyclic C₁, C₂, C₃, C₄ and C₅ alkyl functionalities; C₂-C₅ alkenyl includes straight, branched, and where appropriate cyclic C₂, C₃, C₄ and C₅ alkenyl functionalities; C₁-C₅ alkoxy includes straight, branched, and where appropriate cyclic C₁, C₂, C₃, C₄ and C₅ alkoxy functionalities; C₂-C₅ alkenoxy includes straight, branched, and where appropriate cyclic C₂, C₃, C₄ and C₅ alkenoxy functionalities; C₂-C₅ alkynyl includes straight, branched and where appropriate cyclic C₁, C₂, C₃, C₄ and C₅ alkynyl functionalities; and C₂-C₅ alkynoxy includes straight, branched, and where appropriate cyclic C₂, C₃, C₄ and C₅ alkynoxy functionalities.

The term “lower alkyl”, as used herein, and unless otherwise specified, refers to a C₁ to C₄ saturated straight, branched, or if appropriate, a cyclic (for example, cyclopropyl) alkyl group, optionally including substituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The term “alkenyl” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂-C₈)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-ethenyl)-pentenyl. An alkenyl group can be unsubstituted or substituted with one or more suitable substituents.

The term “alkynyl” means a monovalent, unbranched or branched hydrocarbon chain having one or more triple bonds therein. The double bond of an alkynyl group can be unconjugated or conjugated to another unsaturated group. Suitable alkenyl groups include, but are not limited to (C₂-C₈)alkynyl groups, such as ethynyl, propynyl, butynyl, pentynyl, hexynyl, 2-ethylhexynyl, 2-propyl-2-butynyl, 4-(2-methyl-3-ethynyl)-pentynyl. An alkynyl group can be unsubstituted or substituted with one or more suitable substituents.

The term “alkylamino” or “arylamino” refers to an amino group that has one or two alkyl or aryl substituents, respectively.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term “aryl”, as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “alkaryl” or “alkylaryl” refers to an alkyl group with an aryl substituent.

The term aralkyl or arylalkyl refers to an aryl group with an alkyl substituent.

The term “halo”, as used herein, includes chloro, bromo, iodo, and fluoro.

The term “haloalkyl” refers an alkyl group which is substituted by at least one halo group, for example CF₃.

The term “acyl” refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The term “heteroalkyl” refers to an alkyl group substituted by a heteroatom functionality, for example aminoalkyl, alkoxyalkyl, thioalkyl. A heteroalkyl can also refer to an alkyl group which includes a heteroatom in the alkyl chain.

The term “heteroatom” refers to any atom that is not carbon or hydrogen, for example nitrogen, oxygen, sulfur, phosphorus, boron, chlorine, bromine, or iodine. The term “pharmaceutically acceptable salt, ester or prodrug” is used throughout the specification to describe any pharmaceutically acceptable form (such as an ester, phosphate ester, salt of an ester or a related group) of a compound which, upon administration to a patient, provides the compound described in the specification. Pharmaceutically acceptable salts include those derived from pharmaceutically acceptable inorganic or organic bases and acids such as hydrochloric acid, hydrobromic acid, sulfuric acid, nitric acid, phosphoric acid, methanesulfonic acid, ethanesulfonic acid, p-toluensulfonic acid, salicylic acid, malic acid, maleic acid, succinic acid, tartaric acid, citric acid and the like. Suitable salts include those derived from alkali metals such as potassium and sodium, alkaline earth metals such as calcium and magnesium, among numerous other acids well known in the art.

Pharmaceutically acceptable “prodrugs” refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The term “heterocyclic” or “heterocycle” refers to a nonaromatic cyclic group that may be partially (contains at least one double bond) or fully saturated and wherein there is at least one heteroatom, such as oxygen, sulfur, nitrogen, or phosphorus in the ring, and wherein said “heterocyclic” or “heterocycle” group can be optionally substituted with one or more substituent selected from the group consisting of halogen, haloalkyl, alkyl, alkoxy, hydroxy, carboxyl derivatives, amido, hydroxyl, acyl, amino, alkylamino, dialkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991, hereby incorporated by reference.

The term “heteroaryl” or “heteroaromatic”, as used herein, refers to an aromatic that includes at least one sulfur, oxygen, nitrogen or phosphorus in the aromatic ring. Nonlimiting examples of heterocyclics and heteroaromatics are pyrrolidinyl, tetrahydrofuryl, piperazinyl, piperidinyl, morpholino, thiomorpholino, tetrahydropyranyl, imidazolyl, pyrrolinyl, pyrazolinyl, indolinyl, dioxolanyl, or 1,4-dioxanyl, aziridinyl, furyl, furanyl, pyridyl, pyrimidinyl, benzoxazolyl, 1,2,4-oxadiazolyl, 1,3,4-oxadiazolyl, 1,3,4-thiadiazole, indazolyl, 1,3,5-triazinyl, thienyl, tetrazolyl, benzofuranyl, quinolyl, isoquinolyl, benzothienyl, isobenzofuryl, indolyl, isoindolyl, benzimidazolyl, purine, carbazolyl, oxazolyl, thiazolyl, benzothiazolyl, isothiazolyl, 1,2,4-thiadiazolyl, isooxazolyl, pyrrolyl, quinazolinyl, cinnolinyl, phthalazinyl, xanthinyl, hypoxanthinyl, pyrazole, 1,2,3-triazole, 1,2,4-triazole, 1,2,3-oxadiazole, thiazine, pyridazine, benzothiophenyl, isopyrrole, thiophene, pyrazine, or pteridinyl wherein said heteroaryl or heterocyclic group can be optionally substituted with one or more substituent selected from the group consisting of halogen, haloalkyl, alkyl, alkoxy, hydroxy, carboxyl derivatives, amido, hydroxyl, acyl, amino, alkylamino, dialkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991, hereby incorporated by reference.

Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acycl groups such as acetyl and propionyl, methanesulfonyl, and p-toluenelsulfonyl.

As used herein, unless the context suggests otherwise, the term “isotope” in relation to a chemical composition refers to the existence of the isotope in excess of that generally found nature, i.e., in excess of its natural abundance.

### Imaging Agents

Compounds or imaging agents are provided for the imaging of the expression of the CXCR4 receptor. In various embodiments, the compounds are provided that may be used diagnostically in formulations or medicaments for the diagnosis, staging, and treatment-monitoring of chemokine mediated pathologies. In certain embodiments, imaging compositions and methods of imaging a CXCR4 mediated pathology, or a pathology mediated by a CXCR4 chemokine receptor, in a host in need of such treatment, by administering to the host a detectably effective amount of a labeled CXCR4 antagonist, or a pharmaceutically acceptable salt thereof are provided. Exemplary CXCR4 mediated pathologies or pathologies mediated by a CXCR4 receptor include, but are not limited to, cancer, tumors, angiogenesis, metastasis of a tumor/cancer, inflammation, or any other disease, particularly hyperproliferative diseases, involving CXCR4. The imaging agents provided herein offer various advantages or improvements over current imaging agents, particularly with respect to the expression of CXCR4 receptor associated with cancer or inflammation. The imaging agents can be used in various imaging methods to predict metastatic potential before it occurs. The imaging agents can be used in various imaging methods to detect inflammation at its onset and throughout the disease progression. The imaging agents can be used to monitor therapy for any of the disease described herein.

Compounds described herein have the capacity to interact with and potentially inhibit CXCR4 receptor activation. Exemplary compounds have increased bioavailability and efficacy in inhibiting CXCR4 receptors and CXCL12-dependent signaling over known CXCR4 antagonists. These compounds may be used to image metastasis through their capacity to inhibit CXCL12-CXCR4 interactions.

The imaging compounds described herein for imaging chemokine related conditions include radiolabeled CXCR4 antagonists suitable for use in imaging technologies such as a gamma camera, a PET apparatus, a SPECT apparatus, and the like. In a particular embodiment, the radiolabeled CXCR4 antagonist binds to the CXCL12 binding site of CXCR4 protein. Some exemplary embodiments of non-radioactive elements and their radioactive counterparts that can be used as labels in the imaging probes of the present disclosure include, but are not limited to, F-19 (F-18), C-12 (C-11), I-127 (I-125, I-124, I-131, I-123), Cl-36 (Cl-32, Cl-33, Cl-34), Br-80 (Br-74, Br-75, Br-76, Br-77, Br-78), Re-185/187 (Re-186, Re-188), Y-89 (Y-90, Y-86), Lu-177, and Sm-153. Preferred imaging probes of the present disclosure are labeled with one or more radioisotopes, preferably including ¹¹C, ¹⁸F, ⁷⁶Br, ¹²³I, ¹²⁴I, ¹²⁵I, or ¹³¹I, and more preferably ¹⁸F, ⁷⁶Br, ¹²³I, ¹²⁴I, ¹²⁵I, or ¹³¹I are suitable for use in peripheral medical facilities and PET clinics. In particular embodiments, the PET isotope can include, but is not limited to ¹⁸F, ⁶⁴Cu, ²³I, ¹²⁴I, ¹²⁵I, ¹³¹I, ⁷⁶Br, ⁸⁶Y, ⁸⁹Zr, and ⁶⁸Ga. In an exemplary embodiment, the PET isotope is ¹⁸F. Radiolabeled CXCR4 antagonists described herein bind to the CXCL12 binding site of CXCR4 protein and can be detected with a PET scanner.

In a first principal embodiment, a compound of Formula I, or a pharmaceutically acceptable salt, ester or prodrug thereof, is provided for the imaging or detection of a disorder associated with CXCR4 receptor activation, including cancer metastasis and inflammation:

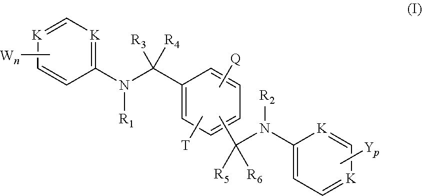

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN; each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl, aralkyl, aryl, heteroarylalkyl and heterocyclylalkyl;
wherein at least one of Q, T, W and Y is a radioisotope selected from the group consisting of ¹⁸F, ⁷⁶Br, ¹²³I, ¹²⁴I, ¹²⁵I, or ¹³¹I;
n is 0, 1, 2 or 3;
p is 0, 1, 2 or 3;
R¹, R², R³, R⁴, R⁵ and R⁶ are independently selected from H, straight chain, branched or cyclic alkyl, alkenyl, alkynyl, aralkyl, aryl heteroaryl, acyl (RC—) and imidoyl (RC(NH)— or RC(NR′)—) groups.

In another embodiment, a compound of Formula I, or a pharmaceutically acceptable salt, ester or prodrug thereof, is provided for imaging of a proliferative disorder, for example metastatic cancer.

In one embodiment of Formula I, one of Q, T, W and Y is a radioisotope selected from the group consisting of ¹⁸F, ⁷⁶Br, ¹²³I, ¹²⁴I, ¹²⁵I, or ¹³¹I. In one embodiment of Formula I, two or more of Q, T, W and Y is a radioisotope selected from the group consisting of ¹⁸F, ⁷⁶Br, ¹²³I, ¹²⁴I, ¹²⁵I, or ¹³¹I.

In one subembodiment of Formula I, at least one of Q, T, W and Y is ¹⁸F. In one embodiment, at least one of W or Y is ¹⁸F. In one subembodiment, one of Q, T, W and Y is ¹⁸F. In one subembodiment, two or more of Q, T, W and Y is ¹⁸F. In one embodiment, one of W or Y is ¹⁸F. In other embodiments, one W is ¹⁸F. In other embodiments, one Y is ¹⁸F.

In one subembodiment of formula I, each K is independently CH or N. In one embodiment, one K is N. In another embodiment, at least two K are N. In yet another embodiment, at least three K are N and in another embodiment, four K are N.

In a subembodiment of formula I, Y is H. In another subembodiment of formula I, Y is straight chained, branched or cyclic alkyl, heteroalkyl or haloalkyl. In one subembodiment of formula I, Y is straight chained or branched alkyl. In another subembodiment of formula I, Y is F, Cl, Br, or I. In another subembodiment of formula I, Y is NH₂, NHR or NR₂. In a specific embodiment of formula I, Y is NR₂. In one embodiment, Y is CONRR′.

In one embodiment, W is a halogen, including F, Cl, Br and I, or R. In certain embodiments, W is a halogen and Y is a straight chained, branched or cyclic alkyl, heteroalkyl or haloalkyl. In one subembodiment of formula I, at least one of W and Y is a halogen, including F, Cl, Br, I, or R. In another embodiment, both W and Y are a halogen. In a specific embodiment, at least one of W and Y is F and in yet another embodiment, both W and Y are F.

In another embodiment, W is a halogen, including F, Cl, Br and I, or R and Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, W is a halogen, including F, Cl, Br and I, or R and Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, W is a halogen, including F, Cl, Br and I, or R and Y is H, acyl, F, Cl, Br, I, OH, OR, NH₂, CO₂H, CO₂R or CN. In certain subembodiments, Y can be R.

In certain subembodiments, at least one of W or Y is R and R can be F or haloalkyl, for example CF₃.

In a specific embodiment of formula I, R¹ and R² are each H or alkyl and in certain embodiments, are each H.

In a specific embodiment of formula I, R³, R⁴, R⁵ and R⁶ are each H or alkyl and in certain embodiments, at least two, at least three or all four are H.

In a specific embodiment, a compound, method and composition including a compound of formula I-1, or a pharmaceutically acceptable salt, ester or prodrug thereof, is provided:

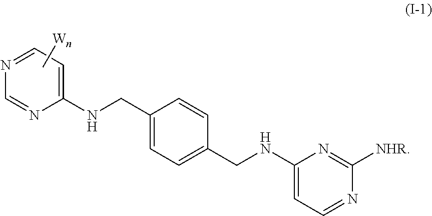

In a subembodiment, n is 2 and 3 and at least one W is ¹⁸F. In another subembodiment, R is alkyl, amino-substituted alkyl, aralkyl, heteroarylalkyl or heterocyclylalkyl. In a particular embodiment, R is heterocyclylalkyl, for example morpholinoalkyl. In another particular embodiment, R is amino-substituted alkyl.

In another embodiment, a compound, method and composition including a compound of formula I-2, or a pharmaceutically acceptable salt, ester or prodrug thereof, is provided:

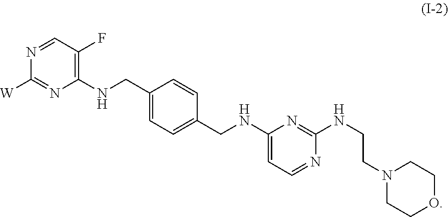

In a particular embodiment, the compound or imaging agent is:

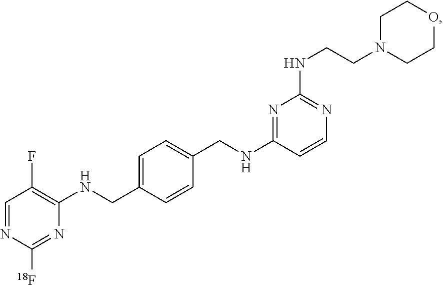

or a pharmaceutically acceptable salt or prodrug thereof.

In a second principal embodiment, a compound of Formula II, or a pharmaceutically acceptable salt, ester or prodrug thereof, is provided for the imaging or detection of a disorder associated with CXCR4 receptor activation, including cancer metastasis and inflammation:

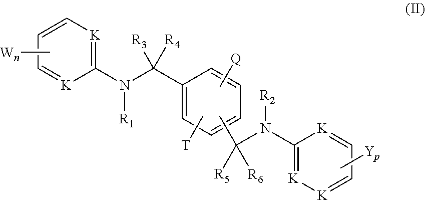

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN; each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl or aralkyl, aryl, heteroarylalkyl and heterocyclylalkyl;
wherein at least one of Q, T, W and Y is a radioisotope selected from the group consisting of ¹⁸F, ⁷⁶Br, ¹²³I, ¹²⁴I, ¹²⁵I, or ¹³¹I;
n is 0, 1, 2 or 3;
p is 0, 1 or 2;
R¹, R², R³, R⁴, R⁵ and R⁶ are independently selected from H, straight chain, branched or cyclic alkyl, alkenyl, alkynyl, aralkyl, aryl heteroaryl, acyl (RC—) and imidoyl (RC(NH)— or RC(NR′)—) groups.

In another embodiment, a compound of Formula II, or a pharmaceutically acceptable salt, ester or prodrug thereof, is provided for imaging of a proliferative disorder, for example metastatic cancer.

In one embodiment of Formula II, one of Q, T, W and Y is a radioisotope selected from the group consisting of ¹⁸F, ⁷⁶Br, ¹²³I, ¹²⁴I, ¹²⁵I, or ¹³¹I. In one embodiment of Formula II, two or more of Q, T, W and Y is a radioisotope selected from the group consisting of ¹⁸F, ⁷⁶Br, ¹²³I, ¹²⁴I, ¹²⁵I, or ¹³¹I.

In one subembodiment of Formula II, at least one of Q, T, W and Y is ¹⁸F. In one embodiment, at least one of W or Y is ¹⁸F. In one subembodiment, one of Q, T, W and Y is ¹⁸F. In one subembodiment, two or more of Q, T, W and Y is ¹⁸F. In one embodiment, one of W or Y is ¹⁸F. In other embodiments, one W is ¹⁸F. In other embodiments, one Y is ¹⁸F.

In one subembodiment of Formula II, each K is independently CH or N. In one embodiment, one K is N. In another embodiment, at least two K are N. In yet another embodiment, at least three K are N, in another embodiment, four K are N and in yet another embodiment, five K are N.

In one embodiment, the compound is of the formula II-a, II-b or II-c:

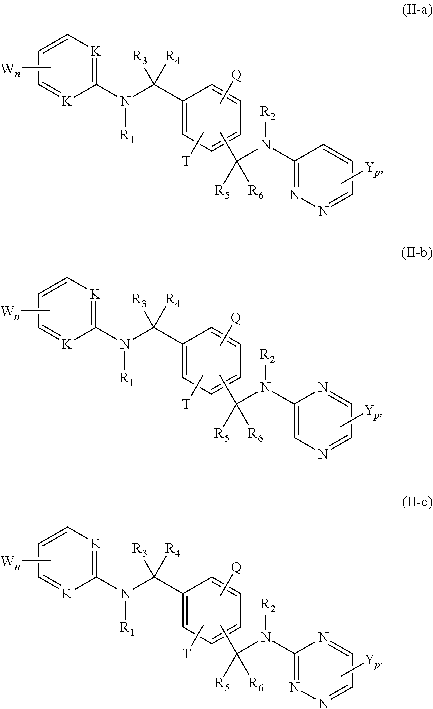

In a subembodiment of formula II, Y is H. In another subembodiment of formula II, Y is straight chained, branched or cyclic alkyl, heteroalkyl or haloalkyl. In one subembodiment of formula II, Y is straight chained or branched alkyl. In another subembodiment of formula II, Y is F, Cl, Br, or I. In yet another subembodiment of formula I, Y is NH₂, NHR or NR₂. In one subembodiment, Y is acyl. In another subembodiment of formula I, Y′ is F, Cl, Br, or I. In yet another subembodiment, Y is NH₂, NHR or NR₂. In a specific embodiment, Y is OR. In certain embodiments, at least one Y is NR₂ and another Y is OR or H. In certain embodiments, R is heteroalkyl and in specific embodiments, the heteroatom is O or N. In certain subembodiments, Y is CONRR′.

In one embodiment, W is a halogen, including F, Cl, Br and I, or R. In certain embodiments, W is a halogen and Y is a straight chained, branched or cyclic alkyl, heteroalkyl or haloalkyl. In one subembodiment of formula II, at least one of W and Y is a halogen, including F, Cl, Br, I, or R. In another embodiment, both W and Y are a halogen. In a specific embodiment, at least one of W and Y is F and in yet another embodiment, both W and Y are F.

In another embodiment, W is a halogen, including F, Cl, Br and I, or R and Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, W is a halogen, including F, Cl, Br and I, or R and Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, W is a halogen, including F, Cl, Br and I, or R and Y is H, acyl, F, Cl, Br, I, OH, OR, NH₂, CO₂H, CO₂R or CN. In certain subembodiments, Y can be R.

In certain subembodiments, at least one of W or Y is R and R can be F or haloalkyl, for example CF₃.

In a specific embodiment of formula II, R¹ and R² are each H or alkyl and in certain embodiments, are each H.

In a specific embodiment of formula II, R³, R⁴, R⁵ and R⁶ are each H or alkyl and in certain embodiments, at least two, at least three or all four are H.

In a third principal embodiment, a compound of Formula III, or a pharmaceutically acceptable salt, ester or prodrug thereof, is provided for the imaging or detection of a disorder associated with CXCR4 receptor activation, including cancer metastasis and inflammation:

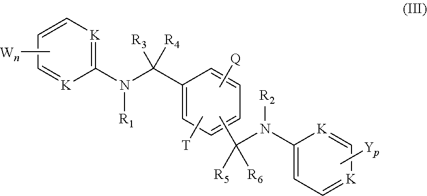

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl or aralkyl, aryl, heteroarylalkyl and heterocyclylalkyl;
wherein at least one of Q, T, W and Y is a radioisotope selected from the group consisting of ¹⁸F, ⁷⁶Br, ¹²³I, ¹²⁴I, ¹²⁵I, or ¹³¹I;
n is 0, 1, 2 or 3;
p is 0, 1 or 2;
R¹, R², R³, R⁴, R⁵ and R⁶ are each independently selected from H, straight chain, branched or cyclic alkyl, alkenyl, alkynyl, aralkyl, aryl heteroaryl, acyl (RC—) and imidoyl (RC(NH)— or RC(NR′)—) groups.

In another embodiment, a compound of Formula III, or a pharmaceutically acceptable salt, ester or prodrug thereof, is provided for imaging of a proliferative disorder, for example metastatic cancer.

In one embodiment of Formula III, one of Q, T, W and Y is a radioisotope selected from the group consisting of ¹⁸F, ⁷⁶Br, ¹²³I, ¹²⁴I, ¹²⁵I, or ¹³¹I. In one embodiment of Formula III, two or more of Q, T, W and Y is a radioisotope selected from the group consisting of ¹⁸F, ⁷⁶Br, ¹²³I, ¹²⁴I, ¹²⁵I, or ¹³¹I.

In one subembodiment of Formula III, at least one of Q, T, W and Y is ¹⁸F. In one embodiment, at least one of W or Y is ¹⁸F. In one subembodiment, one of Q, T, W and Y is ¹⁸F. In one subembodiment, two or more of Q, T, W and Y is ¹⁸F. In one embodiment, one of W or Y is ¹⁸F. In other embodiments, one W is ¹⁸F. In other embodiments, one Y is ¹⁸F.

In one subembodiment of formula III, each K is independently CH or N. In one embodiment, one K is N. In another embodiment, at least two K are N. In yet another embodiment, at least three K are N and in another embodiment, four K are N.

In a subembodiment of formula III, at least one Y is H. In another subembodiment of formula III, at least one Y is straight chained, branched or cyclic alkyl, heteroalkyl or haloalkyl. In one subembodiment of formula III, Y is straight chained or branched alkyl.

In another subembodiment of formula III, at least one Y is F, Cl, Br, or I. In yet another subembodiment of formula III, at least one Y is NH₂, NHR or NR₂. In a specific embodiment of formula III, at least one Y is NR₂.

In one embodiment, at least one W is a halogen, including F, Cl, Br and I, or R. In certain embodiments, at least one W is a halogen, and at least one Y is a straight-chain, branched or cyclic alkyl, heteroalkyl or haloalkyl. In one subembodiment of formula III, at least one of W and Y is a halogen, including F, Cl, Br, I, or R. In another embodiment, at least one W and at least one Y are a halogen. In a specific embodiment, at least one of W and Y is F and in yet another embodiment, at least one W and at least one Y are F.

In another embodiment, at least one W is a halogen, including F, Cl, Br and I, or R and at least one Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, at least one W is a halogen, including F, Cl, Br and I, or R and at least one Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, at least one of W and W′ is a halogen, including F, Cl, Br and I, or R and at least one Y is H, acyl, F, Cl, Br, I, OH, OR, NH₂, CO₂H, CO₂R or CN. In certain subembodiments, at least one Y is R. In certain subembodiments, at least one of W and Y is R and R can be F or haloalkyl, for example CF₃.

In a specific embodiment of formula III, R¹ and R² are each H or alkyl and in certain embodiments, are each H.

In a specific embodiment of formula III, R³, R⁴, R⁵ and R⁶ are each H or alkyl and in certain embodiments, at least two, at least three or all four are H.

In a fourth principal embodiment, a compound of Formula IV, or a pharmaceutically acceptable salt, ester or prodrug thereof, is provided for the imaging or detection of a disorder associated with CXCR⁴ receptor activation, including cancer metastasis and inflammation:

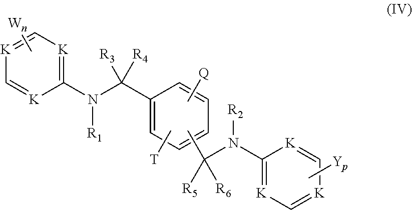

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl or aralkyl, aryl, heteroarylalkyl and heterocyclylalkyl;
wherein at least one of Q, T, W and Y is a radioisotope selected from the group consisting of ¹⁸F, ⁷⁶Br, ¹²³I, ¹²⁴I, ¹²⁵I, or ¹³¹I;
n is 0, 1 or 2;
p is 0, 1 or 2;
R¹, R², R³, R⁴, R⁵ and R⁶ are each independently selected from H, straight chain, branched or cyclic alkyl, alkenyl, alkynyl, aralkyl, aryl heteroaryl, acyl (RC—) and imidoyl (RC(NH)— or RC(NR′)—) groups.

In another embodiment, a compound of Formula IV, or a pharmaceutically acceptable salt, ester or prodrug thereof, is provided for imaging of a proliferative disorder, for example metastatic cancer.

In one embodiment of Formula IV, one of Q, T, W and Y is a radioisotope selected from the group consisting of ¹⁸F, ⁷⁶Br, ¹²³I, ¹²⁴I, ¹²⁵I, or ¹³¹I. In one embodiment of Formula IV, two or more of Q, T, W and Y is a radioisotope selected from the group consisting of ¹⁸F, ⁷⁶Br, ¹²³I, ¹²⁴I, ¹²⁵I, or ¹³¹I.

In one subembodiment of Formula IV, at least one of Q, T, W and Y is ¹⁸F. In one embodiment, at least one of W or Y is ¹⁸F. In one subembodiment, one of Q, T, W and Y is ¹⁸F. In one subembodiment, two or more of Q, T, W and Y is ¹⁸F. In one embodiment, one of W or Y is ¹⁸F. In other embodiments, one W is ¹⁸F. In other embodiments, one Y is ¹⁸F.

In one subembodiment of formula IV, each K is independently CH or N. In one embodiment, one K is N. In another embodiment, at least two K are N. In yet another embodiment, at least three K are N, in another embodiment, four K are N and in yet another embodiment, five K are N. In one embodiment, the compound is of the formula IV-a:

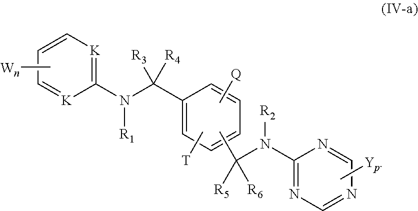

In a subembodiment of formula IV, at least one Y is H. In another subembodiment of formula IV, at least one Y is straight chained, branched or cyclic alkyl, heteroalkyl or haloalkyl. In one subembodiment of formula IV, at least one Y is straight chained or branched alkyl. In another subembodiment of formula IV, at least one Y is F, Cl, Br, or I. In another subembodiment of formula IV, Y is NH₂, NHR or NR₂. In a specific embodiment of formula IV, Y is NR₂. In certain embodiments, one Y is NR₂ and another Y is OR or H. In certain embodiments, R is heteroalkyl and in specific embodiments, the heteroatom is O or N.

In one embodiment, at least one W is a halogen, including F, Cl, Br and I, or R. In certain embodiments, at least one W is a halogen and at least one Y is a straight chained, branched or cyclic alkyl, heteroalkyl or haloalkyl. In one subembodiment of formula IV, at least one of W and Y is a halogen, including F, Cl, Br, I, or R. In another embodiment, at least one W and at least one Y are a halogen. In a specific embodiment, at least one of W and Y is F and in yet another embodiment, at least one W and at least one Y are F.

In another embodiment, at least one W is a halogen, including F, Cl, Br and I, or R and Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, W at least one is a halogen, including F, Cl, Br and I, or R and Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, at least one W is a halogen, including F, Cl, Br and I, or R and Y is H, acyl, F, Cl, Br, I, OH, OR, NH₂, CO₂H, CO₂R or CN. In certain subembodiments, at least one Y is R.

In certain subembodiments, at least one W or Y is R and R can be F or haloalkyl, for example CF₃.

In a specific embodiment of formula IV, R¹ and R² are each H or alkyl and in certain embodiments, are each H.

In a specific embodiment of formula IV, R³, R⁴, R⁵ and R⁶ are each H or alkyl and in certain embodiments, at least two, at least three or all four are H.

In a fifth principal embodiment, a compound of Formula V, or a pharmaceutically acceptable salt, ester or prodrug thereof, is provided for the imaging or detection of a disorder associated with CXCR⁴ receptor activation, including cancer metastasis and inflammation:

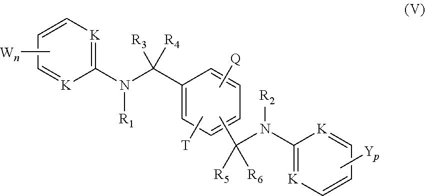

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl or aralkyl, aryl, heteroarylalkyl and heterocyclylalkyl;

wherein at least one of Q, T, W and Y is a radioisotope selected from the group consisting of ¹⁸F, ⁷⁶Br, ¹²³I, ¹²⁴I, ¹²⁵I, or ¹³¹I;
n is 0, 1, 2 or 3;
p is 0, 1, 2 or 3;
R¹, R², R³, R⁴, R⁵ and R⁶ are each independently selected from H, straight chain, branched or cyclic alkyl, alkenyl, alkynyl, aralkyl, aryl, heteroaryl, heterocycle, acyl (RC—) and imidoyl (RC(NH)— or RC(NR′)—) groups.

In another embodiment, a compound of Formula V, or a pharmaceutically acceptable salt, ester or prodrug thereof, is provided for imaging of a proliferative disorder, for example metastatic cancer.

In one embodiment of Formula V, one of Q, T, W and Y is a radioisotope selected from the group consisting of ¹⁸F, ⁷⁶Br, ¹²³I, ¹²⁴I, ¹²⁵I, or ¹³¹I. In one embodiment of Formula V, two or more of Q, T, W and Y is a radioisotope selected from the group consisting of ¹⁸F, ⁷⁶Br, ¹²³I, ¹²⁴I, ¹²⁵I, or ¹³¹I.

In one subembodiment of Formula V, at least one of Q, T, W and Y is ¹⁸F. In one embodiment, at least one of W or Y is ¹⁸F. In one subembodiment, one of Q, T, W and Y is ¹⁸F. In one subembodiment, two or more of Q, T, W and Y is ¹⁸F. In one embodiment, one of W or Y is ¹⁸F. In other embodiments, one W is ¹⁸F. In other embodiments, one Y is ¹⁸F.

In one subembodiment of formula V, each K is independently CH or N. In one embodiment, one K is N. In another embodiment, at least two K are N. In yet another embodiment, at least three K are N. In a particular embodiment, four K are N. In another embodiment, at least one K is CX. In a particular embodiment, four K are N.

In a subembodiment of formula V, Y is H. In another subembodiment of formula V, Y is straight chained, branched or cyclic alkyl, heteroalkyl or haloalkyl. In one subembodiment of formula V, Y is straight chained or branched alkyl. In one embodiment, Y is haloalkyl, for example CF₃. In another subembodiment of formula V, Y is F, Cl, Br, or I. In yet another subembodiments of formula V, Y is NH₂, NHR or NR₂. In a specific embodiment of formula V, Y is NR₂. In one embodiment, Y is CONRR′. In a specific embodiment, Y is C(O)-heterocycle, wherein the heterocycle may be unsubstituted or substituted by hydroxy, alkyl or alkoxyalkyl. In a particular embodiment,

### Y is

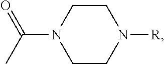

for example

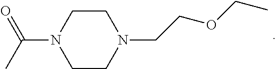

In another embodiment, Y is

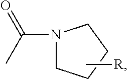

for example

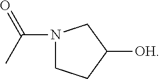

In one embodiment, n is 0. In another embodiment, p is 0. In one embodiment, n is 1. In another embodiment, p is 1. In one embodiment, n is 2. In another embodiment, p is 2. In a particular embodiment, one Y is C(O)-heterocycle, wherein the heterocycle may be unsubstituted or substituted by hydroxy, alkyl or alkoxyalkyl, and another Y is haloalkyl. In a subembodiment, one Y is

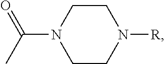

for example

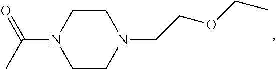

and another Y is haloalkyl, for example CF₃. In another subembodiment, one Y is

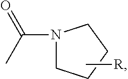

for example

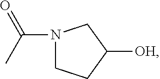

and another Y is haloalkyl, for example CF₃.

In one embodiment, W is H. In another embodiment, W is a halogen, including F, Cl, Br and I, or R. In certain embodiments, W is a halogen and Y is a straight chained, branched or cyclic alkyl, heteroalkyl or haloalkyl. In a particular embodiment, at least one W is halo, for example F or Cl, and at least one Y is a haloalkyl, for example CF₃.

In another embodiment, W is a halogen, including F, Cl, Br and I, or R and Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, W is a halogen, including F, Cl, Br and I, or R and Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, W is a halogen, including F, Cl, Br and I, or R and Y is H, acyl, F, Cl, Br, I, OH, OR, NH₂, CO₂H, CO₂R or CN. In certain subembodiments, Y can be R.

In certain subembodiments, at least one of W or Y is R and R can be F or haloalkyl, for example CF₃.

In a particular embodiment, W is H and at least one Y is not H. In another particular embodiment, Y is H and at least one W is not H.

In a specific embodiment of formula V, R¹ and R² are each independently H or alkyl. In another embodiment, R¹ and R² are each H.

In a specific embodiment of formula V, R³, R⁴, R⁵ and R⁶ are each independently H or alkyl. In another embodiment, at least two, at least three or all four of R³, R⁴, R⁵ and R⁶ are H.

In one subembodiment of any of the foregoing formulae, R¹, R², R³, R⁴, R⁵, and R⁶ are each H, each K is N.

In one embodiment, the compound is a compound of Formula (I)-(V) or a compound wherein compounds wherein a 6-membered aromatic ring is substituted by two CR₂—NR-aryl or CR₂—NR-heteroaryl groups, and wherein the 6-membered aromatic ring, aryl or heteroaryl groups may be optionally substituted with Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, heteroalkyl, heterocycle, haloalkyl or aralkyl groups, aryl and heteroaryl.

In another particular embodiment, a method of imaging or detecting metastasis of a malignant cell is provided that includes contacting the cells with a compound of Formula (I)-(V) as described above, or a pharmaceutically acceptable salt, ester or prodrug thereof.

In another embodiment, CXCR4 antagonists for non-invasive imaging techniques such as magnetic resonance spectroscopy (MRS) or magnetic resonance imaging (MRI) are provided. CXCR4 antagonists suitable for MRS or MRI are labeled with isotopes detectable by nuclear magnetic resonance spectroscopy, for example ¹³C. CXCR4 antagonists of Formulas VI-X described herein may be isotopically labeled, for example with ¹³C, and used as in vivo or in vitro imaging agents for MRS or MRI imaging.

Optical molecular imaging is an attractive modality that has been employed recently in many aspects of biomedical research aiming at using light to detect cellular and molecular events in vivo. This targeted imaging technique largely relies on near-infrared (NIR) dyes that emit light in the NIR window (700-900 nm). Imaging in this range is of importance because of the increased tissue penetration and reduced absorption by physiologically abundant molecules such as hemoglobin (600 nm) and water (>1200 nm) compared to other wavelengths. Optical dyes operate by absorbing energy at one wavelength but the reemitting light at a longer wavelength. Most NIR dyes belong to the family of cyanine, rhodamine or oxazine organic molecules. NIR dyes conjugated to small molecules have been used successfully to image tumors (see for example Tung C H, et al. A receptor-targeted near-infrared fluorescence probe for in vivo tumor imaging. Chem Bio Chem 2002, 3:784-786; Achilefu S, et al. “Novel receptor-targeted fluorescent contrast agents for in vivo tumor imaging” Invest Radiol 2000, 35:479-485; Becker A, et al. “Receptor-targeted optical imaging of tumors with near-infrared fluorescent ligands” Nat Biotechnol 2001, 19:327-331).

The NIR wavelength range has been reported to image deep tissues (3-5 cm) with much less interference of absorbance, scattering, and autofluorescence. The NIR dye, indocyanine green (ICG; Alcorn Pharmaceuticals, Buffalo Grove, Ill.), is FDA-approved and has been safely used in humans for decades at 5 mg/kg intravenously up to a total maximum of 25 mg for use in hepatic clearance, cardiovascular function, and assessing retinopathy. However, ICG does not have a functional group for conjugation to molecular targeting entities, such as antibodies or their fragments, aptamers, or peptides. Instead, ICG associates with albumin in the blood and provides a unique opportunity to translate NIR instrumentation for lymphatic imaging (Sharma R, et al. “Quantitative imaging of lymph function” Am J Physiol Heart Circ Physiol 2007 June; 292(6):H3109-18; Sevick-Muraca E M, et al. “Imaging of lymph flow in breast cancer patients after microdose administration of a near-infrared fluorophore: feasibility study” Radiology 2008 March; 246(3):734-41). Alternatively, there are commercially available NIR dyes with functional groups for conjugation. IRDye800CW (LI-COR Biosciences) with the emission spectrum centered near 800 nm, has passed animal toxicity studies using a protocol reviewed by the Food and Drug Administration in 2007, resulting in great potential for clinical imaging use.

A dye or near-infrared dye suitable for use in in vivo or in vitro imaging can be linked or attached to the CXCR4 antagonist described herein to provide optical imaging of the expression of CXCR4 receptors or a disorder associated with CXCR4 receptor activation, including cancer metastasis and inflammation.

In another embodiment, CXCR4 antagonists for non-invasive imaging techniques such as optical imaging are provided. CXCR4 antagonists suitable for optical imaging are labeled with dyes or dye tags, for example near-infrared dyes. Exemplary near infrared dyes that are suitable for in vivo imaging include IRDye680 and IRDye800CW. CXCR4 antagonists of Formulas VI-X described herein may be labeled with dyes or dye tags and used as in vivo or in vitro imaging agents for optical imaging.

A compound of Formula VI, or a pharmaceutically acceptable salt, ester or prodrug thereof, labeled with isotopes or dyes for the imaging or detection of a disorder associated with CXCR4 receptor activation, including cancer metastasis and inflammation is provided:

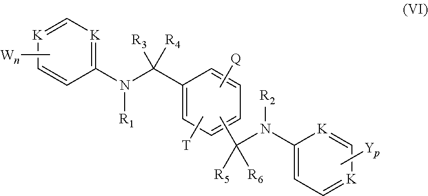

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl, aralkyl, aryl, heteroarylalkyl and heterocyclylalkyl;
n is 0, 1, 2 or 3;
p is 0, 1, 2 or 3;
R¹, R², R³, R⁴, R⁵ and R⁶ are independently selected from H, straight chain, branched or cyclic alkyl, alkenyl, alkynyl, aralkyl, aryl heteroaryl, acyl (RC—) and imidoyl (RC(NH)— or RC(NR′)—) groups.

In one subembodiment of formula VI, each K is independently CH or N. In one embodiment, one K is N. In another embodiment, at least two K are N. In yet another embodiment, at least three K are N and in another embodiment, four K are N.

In a subembodiment of formula VI, Y is H. In another subembodiment, Y is straight chained, branched or cyclic alkyl, heteroalkyl or haloalkyl. In one subembodiment, Y is straight chained or branched alkyl. In another subembodiment, Y is F, Cl, Br, or I. In yet another subembodiments, Y is NH₂, NHR or NR₂. In a specific embodiment, Y is NR₂. In one embodiment, Y is CONRR′.

In one embodiment, W is a halogen, including F, Cl, Br and I, or R. In certain embodiments, W is a halogen and Y is a straight chained, branched or cyclic alkyl, heteroalkyl or haloalkyl. In one subembodiment, at least one of W and Y is a halogen, including F, Cl, Br, I, or R. In another embodiment, both W and Y are a halogen. In a specific embodiment, at least one of W and Y is F and in yet another embodiment, both W and Y are F.

In another embodiment, W is a halogen, including F, Cl, Br and I, or R and Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, W is a halogen, including F, Cl, Br and I, or R and Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, W is a halogen, including F, Cl, Br and I, or R and Y is H, acyl, F, Cl, Br, I, OH, OR, NH₂, CO₂H, CO₂R or CN. In certain subembodiments, Y can be R.

In certain subembodiments, at least one of W or Y is R and R can be F or haloalkyl, for example CF₃.

In a specific embodiment, R¹ and R² are each H or alkyl and in certain embodiments, are each H.

In a specific embodiment, R³, R⁴, R⁵ and R⁶ are each H or alkyl and in certain embodiments, at least two, at least three or all four are H.

In a specific embodiment, a compound, method and composition including a compound of formula VI-1, or a pharmaceutically acceptable salt, ester or prodrug thereof, is provided:

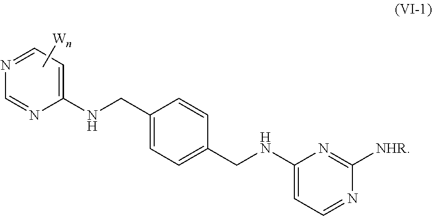

In a subembodiment, n is 2 and 3. In another subembodiment, R is alkyl, amino-substituted alkyl, aralkyl, heteroarylalkyl or heterocyclylalkyl. In a particular embodiment, R is heterocyclylalkyl, for example morpholinoalkyl. In another particular embodiment, R is amino-substituted alkyl.

In another embodiment, a compound, method and composition including a compound of formula VI-2, or a pharmaceutically acceptable salt, ester or prodrug thereof, is provided:

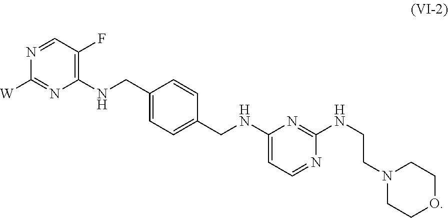

A compound of Formula VII, or a pharmaceutically acceptable salt, ester or prodrug thereof, labeled with isotopes or dyes for the imaging or detection of a disorder associated with CXCR4 receptor activation, including cancer metastasis and inflammation is provided:

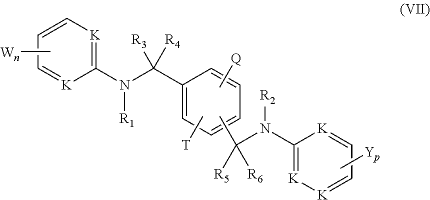

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl or aralkyl, aryl, heteroarylalkyl and heterocyclylalkyl;
n is 0, 1, 2 or 3;
p is 0, 1 or 2;
R¹, R², R³, R⁴, R⁵ and R⁶ are independently selected from H, straight chain, branched or cyclic alkyl, alkenyl, alkynyl, aralkyl, aryl heteroaryl, acyl (RC—) and imidoyl (RC(NH)— or RC(NR′)—) groups.

In one subembodiment of Formula VII, each K is independently CH or N. In one embodiment, one K is N. In another embodiment, at least two K are N. In yet another embodiment, at least three K are N, in another embodiment, four K are N and in yet another embodiment, five K are N.

In one embodiment, the compound is of the formula VII-a, VII-b or VII-c:

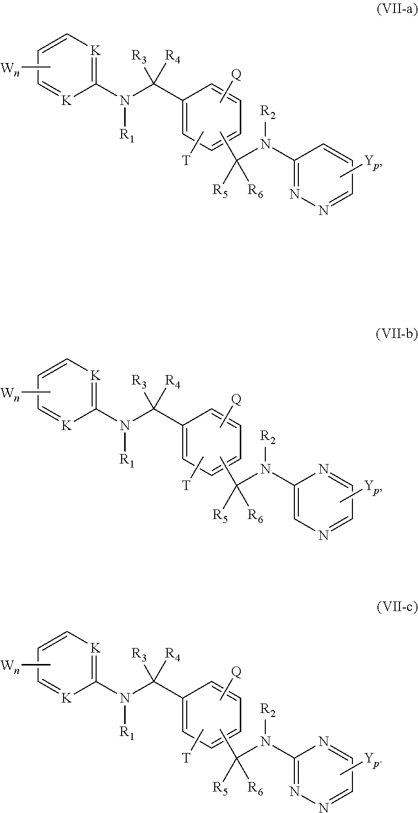

In a subembodiment of formula VII, Y is H. In another subembodiment, Y is straight chained, branched or cyclic alkyl, heteroalkyl or haloalkyl. In one subembodiment of formula VII, Y is straight chained or branched alkyl. In another subembodiment of formula VII, Y is F, Cl, Br, or I. In yet another subembodiment, Y is NH₂, NHR or NR₂. In one subembodiment, Y is acyl. In another subembodiment, Y′ is F, Cl, Br, or I. In yet another subembodiment, Y is NH₂, NHR or NR₂. In a specific embodiment, Y is OR. In certain embodiments, at least one Y is NR₂ and another Y is OR or H. In certain embodiments, R is heteroalkyl and in specific embodiments, the heteroatom is O or N. In certain subembodiments, Y is CONRR′.

In one embodiment, W is a halogen, including F, Cl, Br and I, or R. In certain embodiments, W is a halogen and Y is a straight chained, branched or cyclic alkyl, heteroalkyl or haloalkyl. In one subembodiment, at least one of W and Y is a halogen, including F, Cl, Br, I, or R. In another embodiment, both W and Y are a halogen. In a specific embodiment, at least one of W and Y is F and in yet another embodiment, both W and Y are F.

In another embodiment, W is a halogen, including F, Cl, Br and I, or R and Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, W is a halogen, including F, Cl, Br and I, or R and Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, W is a halogen, including F, Cl, Br and I, or R and Y is H, acyl, F, Cl, Br, I, OH, OR, NH₂, CO₂H, CO₂R or CN. In certain subembodiments, Y can be R.

In certain subembodiments, at least one of W or Y is R and R can be F or haloalkyl, for example CF₃.

In a specific embodiment of formula VII, R¹ and R² are each H or alkyl and in certain embodiments, are each H.

In a specific embodiment of formula VII, R³, R⁴, R⁵ and R⁶ are each H or alkyl and in certain embodiments, at least two, at least three or all four are H.

A compound of Formula VIII, or a pharmaceutically acceptable salt, ester or prodrug thereof, labeled with isotopes or dyes for the imaging or detection of a disorder associated with CXCR⁴ receptor activation, including cancer metastasis and inflammation is provided:

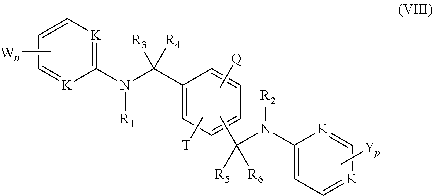

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl or aralkyl, aryl, heteroarylalkyl and heterocyclylalkyl;
n is 0, 1, 2 or 3;
p is 0, 1 or 2;
R¹, R², R³, R⁴, R⁵ and R⁶ are each independently selected from H, straight chain, branched or cyclic alkyl, alkenyl, alkynyl, aralkyl, aryl heteroaryl, acyl (RC—) and imidoyl (RC(NH)— or RC(NR′)—) groups.

In one subembodiment of formula VIII, each K is independently CH or N. In one embodiment, one K is N. In another embodiment, at least two K are N. In yet another embodiment, at least three K are N and in another embodiment, four K are N.

In a subembodiment of formula VIII, at least one Y is H. In another subembodiment, at least one Y is straight chained, branched or cyclic alkyl, heteroalkyl or haloalkyl. In one subembodiment, Y is straight chained or branched alkyl. In another subembodiment, at least one Y is F, Cl, Br, or I. In yet another subembodiment, at least one Y is NH₂, NHR or NR₂. In a specific embodiment, at least one Y is NR₂.

In one embodiment, at least one W is a halogen, including F, Cl, Br and I, or R. In certain embodiments, at least one W is a halogen, and at least one Y is a straight-chain, branched or cyclic alkyl, heteroalkyl or haloalkyl. In one subembodiment, at least one of W and Y is a halogen, including F, Cl, Br, I, or R. In another embodiment, at least one W and at least one Y are a halogen. In a specific embodiment, at least one of W and Y is F and in yet another embodiment, at least one W and at least one Y are F.

In another embodiment, at least one W is a halogen, including F, Cl, Br and I, or R and at least one Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, at least one W is a halogen, including F, Cl, Br and I, or R and at least one Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, at least one of W and W′ is a halogen, including F, Cl, Br and I, or R and at least one Y is H, acyl, F, Cl, Br, I, OH, OR, NH₂, CO₂H, CO₂R or CN. In certain subembodiments, at least one Y is R.

In certain subembodiments, at least one of W and Y is R and R can be F or haloalkyl, for example CF₃.

In a specific embodiment, R¹ and R² are each H or alkyl and in certain embodiments, are each H.

In a specific embodiment, R³, R⁴, R⁵ and R⁶ are each H or alkyl and in certain embodiments, at least two, at least three or all four are H.

A compound of Formula IX, or a pharmaceutically acceptable salt, ester or prodrug thereof, labeled with isotopes or dyes for the imaging or detection of a disorder associated with CXCR⁴ receptor activation, including cancer metastasis and inflammation is provided:

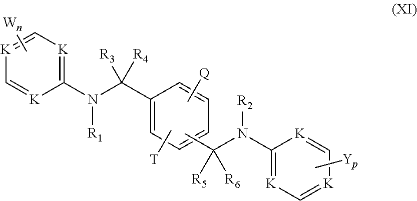

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl or aralkyl, aryl, heteroarylalkyl and heterocyclylalkyl;
n is 0, 1 or 2;
p is 0, 1 or 2;
R¹, R², R³, R⁴, R⁵ and R⁶ are each independently selected from H, straight chain, branched or cyclic alkyl, alkenyl, alkynyl, aralkyl, aryl heteroaryl, acyl (RC—) and imidoyl (RC(NH)— or RC(NR′)—) groups.

In one subembodiment of formula IX, each K is independently CH or N. In one embodiment, one K is N. In another embodiment, at least two K are N. In yet another embodiment, at least three K are N, in another embodiment, four K are N and in yet another embodiment, five K are N. In one embodiment, the compound is of the formula IX-a:

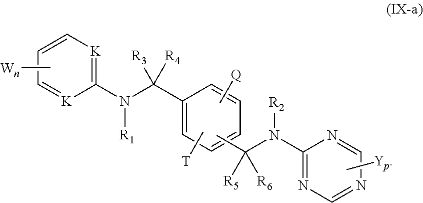

In a subembodiment, at least one Y is H. In another subembodiment, at least one Y is straight chained, branched or cyclic alkyl, heteroalkyl or haloalkyl. In one subembodiment, at least one Y is straight chained or branched alkyl. In another subembodiment, at least one Y is F, Cl, Br, or I. In yet another subembodiments, Y is NH₂, NHR or NR₂. In a specific embodiment, Y is NR₂. In certain embodiments, one Y is NR₂ and another Y is OR or H. In certain embodiments, R is heteroalkyl and in specific embodiments, the heteroatom is O or N.

In one embodiment, at least one W is a halogen, including F, Cl, Br and I, or R. In certain embodiments, at least one W is a halogen and at least one Y is a straight chained, branched or cyclic alkyl, heteroalkyl or haloalkyl. In one subembodiment of formula IV, at least one of W and Y is a halogen, including F, Cl, Br, I, or R. In another embodiment, at least one W and at least one Y are a halogen. In a specific embodiment, at least one of W and Y is F and in yet another embodiment, at least one W and at least one Y are F.

In another embodiment, at least one W is a halogen, including F, Cl, Br and I, or R and Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, W at least one is a halogen, including F, Cl, Br and I, or R and Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, at least one W is a halogen, including F, Cl, Br and I, or R and Y is H, acyl, F, Cl, Br, I, OH, OR, NH₂, CO₂H, CO₂R or CN. In certain subembodiments, at least one Y is R.

In certain subembodiments, at least one W or Y is R and R can be F or haloalkyl, for example CF₃.

In a specific embodiment, R¹ and R² are each H or alkyl and in certain embodiments, are each H.

In a specific embodiment, R³, R⁴, R⁵ and R⁶ are each H or alkyl and in certain embodiments, at least two, at least three or all four are H.

A compound of Formula X, or a pharmaceutically acceptable salt, ester or prodrug thereof, labeled with isotopes or dyes for the imaging or detection of a disorder associated with CXCR⁴ receptor activation, including cancer metastasis and inflammation is provided:

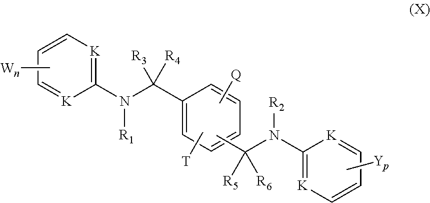

wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl or aralkyl, aryl, heteroarylalkyl and heterocyclylalkyl;
n is 0, 1, 2 or 3;
p is 0, 1, 2 or 3;
R¹, R², R³, R⁴, R⁵ and R⁶ are each independently selected from H, straight chain, branched or cyclic alkyl, alkenyl, alkynyl, aralkyl, aryl, heteroaryl, heterocycle, acyl (RC—) and imidoyl (RC(NH)— or RC(NR′)—) groups.

In one subembodiment of formula X, each K is independently CH or N. In one embodiment, one K is N. In another embodiment, at least two K are N. In yet another embodiment, at least three K are N. In a particular embodiment, four K are N. In another embodiment, at least one K is CX. In a particular embodiment, four K are N.

In a subembodiment of formula X, Y is H. In another subembodiment, Y is straight chained, branched or cyclic alkyl, heteroalkyl or haloalkyl. In one subembodiment, Y is straight chained or branched alkyl. In one embodiment, Y is haloalkyl, for example CF₃. In another subembodiment, Y is F, Cl, Br, or I. In yet another subembodiments, Y is NH₂, NHR or NR₂. In a specific embodiment, Y is NR₂. In one embodiment, Y is CONRR′. In a specific embodiment, Y is C(O)-heterocycle, wherein the heterocycle may be unsubstituted or substituted by hydroxy, alkyl or alkoxyalkyl. In a particular embodiment, Y is

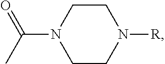

for example

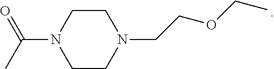

In another embodiment, Y is

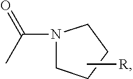

for example

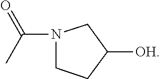

In one embodiment, n is 0. In another embodiment, p is 0. In one embodiment, n is 1. In another embodiment, p is 1. In one embodiment, n is 2. In another embodiment, p is 2. In a particular embodiment, one Y is C(O)-heterocycle, wherein the heterocycle may be unsubstituted or substituted by hydroxy, alkyl or alkoxyalkyl, and another Y is haloalkyl. In a subembodiment, one Y is

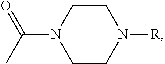

for example

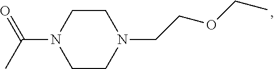

and another Y is haloalkyl, for example CF₃. In another subembodiment, one Y is

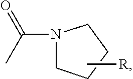

for example

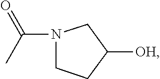

and another Y is haloalkyl, for example CF₃.

In one embodiment, W is H. In another embodiment, W is a halogen, including F, Cl, Br and I, or R. In certain embodiments, W is a halogen and Y is a straight chained, branched or cyclic alkyl, heteroalkyl or haloalkyl. In a particular embodiment, at least one W is halo, for example F or Cl, and at least one Y is a haloalkyl, for example CF₃.

In another embodiment, W is a halogen, including F, Cl, Br and I, or R and Y is NHR, NR₂, NHacyl, N(acyl)₂, and in certain subembodiments, R is selected from straight chain, branched or cyclic alkyl, heteroalkyl, haloalkyl or aralkyl groups. In yet another embodiment, W is a halogen, including F, Cl, Br and I, or R and Y is SR, SR, S₂R, S—NHR, S₂—NHR, S—NRR′ or S₂—NRR′, and in certain subembodiments, R and R′ are selected from straight chain, branched or cyclic alkyl, heteroalkyl, haloalkyl or aralkyl groups. In other embodiments, W is a halogen, including F, Cl, Br and I, or R and Y is H, acyl, F, Cl, Br, I, OH, OR, NH₂, CO₂H, CO₂R or CN. In certain subembodiments, Y can be R.

In certain subembodiments, at least one of W or Y is R and R can be F or haloalkyl, for example CF₃.

In a particular embodiment, W is H and at least one Y is not H. In another particular embodiment, Y is H and at least one W is not H.

In a specific embodiment, R¹ and R² are each independently H or alkyl. In another embodiment, R¹ and R² are each H.

In a specific embodiment, R³, R⁴, R⁵ and R⁶ are each independently H or alkyl. In another embodiment, at least two, at least three or all four of R³, R⁴, R⁵ and R⁶ are H.

In one subembodiment of any of the foregoing formulae, R¹, R², R³, R⁴, R⁵ and R⁶ are each H, each K is N.

In one embodiment, the compound is a compound of Formula (VI)-(X) or a compound wherein compounds wherein a 6-membered aromatic ring is substituted by two CR₂—NR-aryl or CR₂—NR-heteroaryl groups, and wherein the 6-membered aromatic ring, aryl or heteroaryl groups may be optionally substituted with Q, T, W and Y are each independently H, R, acyl, F, Cl, Br, I, OH, OR, NH₂, NHR, NR₂, SR, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, heteroalkyl, heterocycle, haloalkyl or aralkyl groups, aryl and heteroaryl.

In another particular embodiment, a method of imaging or detecting metastasis of a malignant cell is provided that includes contacting the cells with a labeled compound of Formula (VI)-(X) as described above, or a pharmaceutically acceptable salt, ester or prodrug thereof.

In any of the foregoing embodiments, the labeled compound of Formula (VI)-(X) is a compound of Formula (VI)-(X) labeled with a dye, for example a near infrared dye, and can be used as an imaging agent for optical imaging. In particular embodiments, the labeled CXCR4 antagonists can be used for intraoperative imaging.

In any of the foregoing embodiments, the labeled compound of Formula (VI)-(X) is a compound of Formula (VI)-(X) labeled by incorporation of ¹³C into the compound of Formula (VI)-(X), and can be used as an imaging agent for MRS or MRI. In particular embodiments, the carbon atoms in the compound of Formula (VI)-(X) are replaced with ¹³C. In a subembodiment, the ¹³C-labeled CXCR4 antagonist can be detected by hyperpolarized ¹³C MRS.

In certain embodiments, any of the CXCR4 antagonists described herein can be labeled with ¹¹C and detected by PET imaging.

### Methods of Use

The ability to noninvasively and quantitatively image conditions related to over-expression of certain cell surface receptors, such as CXCR4 chemokine receptors using radiolabeled CXCR4 antagonists provides methods of early detection of disease and monitoring of disease progression as well as monitoring the effectiveness of drugs and other treatments. For instance, in the case of CXCR4 receptors that are implicated in cancer as well as indicators of metastatic potential, imaging the expression of these receptors can assist in early and sensitive cancer detection and patient selection for clinical trials based on in vivo expression quantification as well as allow early tumor diagnosis and patient stratification, metastasis prediction and detection, and better treatment monitoring, dose optimization, and the like.

The data provided in the examples below demonstrates that CXCR4/CXCL12 interaction is one of the major requirements for head and neck cancer metastasis. The elevated level of CXCR4 in primary tumors correlates with the metastatic potential of tumors. CXCR4 overexpression has been found in other tumors, such as breast cancer, pancreatic cancer (Koshiba, T. et al. (2000) Clin. Cancer Res. 6: 3530-3535), ovarian epithelial tumors (Scotton, C. J. et al. (2001) Cancer Res. 61: 4961-4965), prostate cancer (Taichman, R. S. (2002) Cancer Res. 62: 1832-1837), kidney cancer (Schrader, A. J. et al. (2002) Br. J. Cancer 86: 1250-1256), and non-small cell lung cancer (Takanami, I. (2003) Int. J. Cancer 105: 186-189).

Accordingly, embodiments of the present disclosure include methods of imaging breast, brain, pancreatic, ovarian, prostate, kidney, head and neck, and non-small lung cancer, among others, as well as methods for detecting/predicting the metastatic potential of such cancers. In particular, metastasis of breast, head and neck, brain, pancreatic, ovarian, prostate, kidney, and non-small lung cancer can be detected and/or predicted by administering a radiolabled CXCR4 antagonist, such as [¹⁸F]M508F, to host in need of such treatment in an effective amount, imaging the host with appropriate imaging technology (e.g., a PET scanner), and detecting the expression of CXCR4 receptors.

The extent of cancerous disease (stage) is a major prognostic factor, and non-invasive staging using imaging technologies has a key role in design of treatment strategies (e.g., surgery vs. radio-chemotherapy vs. adjuvant chemotherapy). The radiolabeled compounds of the present disclosure accumulate in malignant cells to a substantially greater extent than in normal cells and accumulate in highly metastatic cells to a greater extent than in cancer or tumor cells that are not as likely to metastasize. Thus, administration of an imaging compound of the present disclosure is suitable for the identification and imaging of malignant cells and tumors and is further suitable for measuring the stage of tumor development and metastatic potential.

Methods for predicting tumor cell metastasis in a mammal by administering a detectably effective amount of a radiolabeled CXCR4 antagonist, for example [¹⁸F]M508F, or a pharmaceutically acceptable salt or prodrug thereof, and determining the level of expression of CXCR4 receptors by the tumor, wherein a higher level of CXCR4 expression is associated with a greater potential for metastasis are provided. In certain embodiments, the treatment of cancer or metastasis is monitored by tracking the expression of CXCR4 as an indicator of the effectiveness of the treatment.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

In certain embodiments, methods for determining the effectiveness of a drug on various conditions associated with expression (particularly overexpression) of CXCR4 chemokine receptors are provided. Conditions that can be monitored with respect to drug effectiveness include, but are not limited to, inflammation, cancer, tumors, angiogenesis, and metastasis. For instance, the methods of the present disclosure can be used to determine whether a particular drug is effective at inhibiting metastasis in a host having cancer, by monitoring the level of expression of CXCR4 receptors in the host cancer cells, which is an indicator of metastasis and metastatic potential. If expression of CXCR4 receptors decreases with drug treatment, that would indicate that the drug appears to be at least somewhat effective at inhibiting metastasis of the cancer/tumor in the host.

Such methods include administering an amount of a drug to a host; administering a detectably effective amount of a composition including an imaging probe comprising a radiolabeled CXCR4 antagonist, or a pharmaceutically acceptable salt or prodrug thereof, to a host; creating a radiographic image of the location and distribution of the imaging probe in the host with an imaging apparatus; and determining an amount of the imaging probe taken up by host cells wherein the amount of uptake by host mitochondria is related to the effect of the drug on apoptosis in host cells.

Methods of use of the imaging agents provided herein include, but are not limited to: methods of imaging tissue; methods of imaging precancerous tissue, cancer, and tumors; methods of treating precancerous tissue, cancer, and tumors; methods of diagnosing precancerous tissue, cancer, and tumors; methods of monitoring the progress of precancerous tissue, cancer, and tumors; methods of imaging abnormal tissue, and the like. The methods can be used to detect, study, monitor, evaluate, and/or screen, biological events in vivo or in vitro, such as, but not limited to, CXCR4 related biological events.

The imaging agents, compositions, and methods of use provided can be used in vivo or in vitro for imaging cancer cells or tissue; imaging precancerous cells or tissue; diagnosing precancerous tissue, cancer, tumors, and tumor metastases; monitoring the progress and/or staging of precancerous tissue, cancer, and tumors; methods of predicting tumor metastasis; methods of evaluating drug effectiveness on treating and/or preventing cancer, tumors, metastasis, and the like.

In diagnosing and/or monitoring the presence of cancerous cells, precancerous cells, and tumors in a subject, labeled CXCR4 antagonists are administered to the subject in an amount effective to result in uptake of the labeled CXCR4 antagonists into the cells. After administration of the labeled CXCR4 antagonists, cells that take up the labeled CXCR4 antagonists are detected using PET or SPECT imaging. Embodiments of the present disclosure can non-invasively image tissue throughout an animal or patient.

It should be noted that the amount effective to result in uptake of the compound into the cells or tissue of interest will depend upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

Preferred imaging methods provided by the present disclosure include the use of the radiolabeled CXCR4 antagonists of the present disclosure and/or salts thereof that are capable of generating at least a 2:1 target to background ratio of radiation intensity, or more preferably about a 5:1, about a 10:1 or about a 15:1 ratio of radiation intensity between target and background. In certain preferred methods, the radiation intensity of the target tissue is more intense than that of the background. In other embodiments, the present disclosure provides methods where the radiation intensity of the target tissue is less intense than that of the background. Generally, any difference in radiation intensity between the target tissue and the background that is sufficient to allow for identification and visualization of the target tissue is sufficient for use in the methods of the present disclosure.

In preferred methods of the present disclosure, the compounds of the present disclosure are excreted from tissues of the body quickly to prevent prolonged exposure to the radiation of the radiolabeled compound administered to the patient. In particular embodiment, the radiolabeled CXCR4 antagonists provided herein can be used on an outpatient basis. Typically compounds of the present disclosure, including [¹⁸F]M508F and salts thereof, are eliminated from the body in less than about 24 hours. More preferably, compounds of the present disclosure are eliminated from the body in less than about 16 hours, 12 hours, 8 hours, 6 hours, 4 hours, 2 hours, 90 minutes, or 60 minutes.

Preferred imaging agents are stable in vivo such that substantially all, e.g., more than about 50%, 60%, 70%, 80%, or more preferably 90% of the injected compound is not metabolized by the body prior to excretion.

Typical subjects to which compounds of the present disclosure may be administered will be mammals, particularly primates, especially humans. For veterinary applications, a wide variety of subjects will be suitable, e.g. livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine or tissue samples, or blood urine or tissue samples of the animals mentioned for veterinary applications.

Images can be generated by virtue of differences in the spatial distribution of the imaging agents that accumulate at a site having expression, and/or overexpression, of the CXCR4 receptors. The spatial distribution may be measured using any imaging apparatus suitable for the particular label, for example, a gamma camera, a PET apparatus, a SPECT apparatus, MRS, MRI or optical imaging apparatus, and the like. The extent of accumulation of the imaging agent may be quantified using known methods for quantifying radioactive emissions. A particularly useful imaging approach employs more than one imaging agent to perform simultaneous studies. Alternatively, the imaging method may be carried out a plurality of times with increasing administered dose of the pharmaceutically acceptable imaging composition of the present disclosure to perform successive studies using the split-dose image subtraction method, as are known to those of skill in the art.

Preferably, an amount of the imaging agent effective for detection is administered to a subject. A effective amount of the imaging agent may be administered in more than one injection. The effective amount of the imaging agent can vary according to factors such as the degree of susceptibility of the individual, the age, sex, and weight of the individual, idiosyncratic responses of the individual, the dosimetry, and the like. Effective amounts of the imaging agent can also vary according to instrument and film-related factors. Optimization of such factors is well within the level of skill in the art.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

In one embodiment, a method of imaging metastases of a malignant cell is provided that includes administering a compound of at least one of Formula (I)-(V) or Formula (VI)-(X) to a host. The malignant cell can be a tumor cell. In certain embodiments, the compound can be provided to a host before treatment of a tumor. In a separate embodiment, the compound is provided to a patient that has been treated for cancer to reduce the likelihood of recurrence, or reduce mortality associated with a particular tumor. In another embodiment, the compound is administered to a host at high risk of suffering from a proliferative disease. Such high risk can be based, for example, on family history or on a history of exposure to known or presumed carcinogens.

Hosts, including humans suffering from, or at risk for, a proliferative disorder can be treated by administering an effective amount of the imaging agent or a pharmaceutically acceptable prodrug or salt thereof in the presence of a pharmaceutically acceptable carrier or diluent. The imaging agent or composition comprising the imaging agent can be administered by any appropriate route, for example, orally, parenterally, intravenously, intradermally, subcutaneously, or topically, in liquid or solid form. However, the compounds are particularly suited to oral delivery.

A preferred dose of the compound will be in the range from about 1 to 50 mg/kg, preferably 1 to 20 mg/kg, of body weight per day, more generally 0.1 to about 100 mg per kilogram body weight of the recipient per day. The effective dosage range of the pharmaceutically acceptable salts and prodrugs can be calculated based on the weight of the parent compound to be delivered. If the salt, ester or prodrug exhibits activity in itself, the effective dosage can be estimated as above using the weight of the salt, ester or prodrug, or by other means known to those skilled in the art.

In a separate embodiment, a method of imaging or detecting proliferative disorders by administering a compound of Formulas (I)-(V) or a labeled compound of Formula (VI)-(X) to a host is provided. In certain embodiments, the proliferative disorder is cancer, and in particular subembodiments, the disorder is a metastatic cancer. The compounds of the invention can be administered to a host suffering from or at risk of suffering from metastasis of a proliferative disorder, such as cancer. In particular embodiments, the cancer is breast cancer, brain tumor, pancreatic cancer, ovarian tumor, particularly an ovarian epithelial tumor, prostate cancer, kidney cancer, or non-small cell lung cancer.

In another embodiment, the invention provides a method of imaging neovascularization, particularly VEGF-dependent neocascularization, by contacting a cell with a compound of Formula (I)-(V) or a labeled compound of Formula (VI)-(X). The cell can be in a host animal.

In a separate embodiment, a method for imaging diseases of vasculature, inflammatory and degenerative diseases is provided including administering a compound of Formula (I)-(V) or a labeled compound of Formula (VI)-(X) to a host.

The compounds can be used to image or diagnose diseases associated with CXCR4 activity, and in particular of proliferative diseases in any host. However, typically the host is a mammal and more typically is a human. In certain subembodiments the host has been diagnosed with a hyperproliferative disorder prior to administration of the compound, however in other embodiments, the host is merely considered at risk of suffering from such a disorder.

### Compositions and Dosage Forms

Compositions and dosage forms of the disclosure comprise a labeled CXCR4 antagonist described herein, or pharmaceutically acceptable salt, polymorph, solvate, hydrate, dehydrate, co-crystal, anhydrous, or amorphous form thereof. Specific salts of an antagonist of CXCR4 include, but are not limited to, sodium, lithium, potassium salts, and hydrates thereof.

Compositions and unit dosage forms of the disclosure typically also comprise one or more pharmaceutically acceptable excipients or diluents. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines are particularly susceptible to such accelerated decomposition.

Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art.

Unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. In a preferred embodiment, the dosage form is suitable for intravenous administration. In certain embodiment, the dosage is administered to the patient about 0.1 to 36, 0.1 to 24, 0.1 to 2, 0.1 to 1, 10 to 36, or 10 to 24 hours prior to imaging. Examples of dosage forms include, but are not limited to: injection or other intravenous-administration dosage forms; tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. For example, a parenteral dosage form may contain smaller amounts of the active ingredient than an oral dosage form. These and other ways in which specific dosage forms encompassed by this disclosure will vary from one another will be readily apparent to those skilled in the art.

The disclosure further encompasses compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of active ingredient in a dosage form may differ depending on factors such as, but not limited to, the route by which it is to be administered to patients. However, typical dosage forms of the compounds of the disclosure comprise the labeled CXCR4 antagonist, a pharmaceutically acceptable salt, polymorph, solvate, hydrate, dehydrate, co-crystal, anhydrous, or amorphous form thereof, in an amount of from about 10 to 30 mCi of the radionuclide-labeled imaging agent described in combination with a pharmaceutically acceptable carrier. In certain embodiments, the radiolabeled CXCR4 antagonist is administered in a dosage of about 5 to 50 mCi, 5 to 40 mCi, 5 to 30 mCi, 5 to 20 mCi, 10 to 40 mCi, 10 to 30 mCi, or 10 to 20 mCi.

In cases where compounds are sufficiently basic or acidic to form stable nontoxic acid or base salts, administration of the compounds as salts may be appropriate. The term pharmaceutically acceptable salts or complexes refer to salts or complexes that retain the desired biological activity of the compounds of the present invention and exhibit minimal undesired toxicological effects.

Nonlimiting examples of such salts are (a) acid addition salts formed with inorganic acids such as sulfate, nitrate, bicarbonate, and carbonate salts (for example, hydrochloric acid, hydrobromic acid, sulfuric acid, phosphoric acid, nitric acid, and the like), and salts formed with organic acids including tosylate, methanesulfonate, acetate, citrate, malonate, tartarate, succinate, benzoate, ascorbate, α-ketoglutarate, and α-glycerophosphate salts, such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, lithium and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The imaging agent can also be provided as a prodrug, which is converted into a biologically active form in vivo. A prodrug may be converted into the parent drug by various mechanisms, including enzymatic processes and metabolic hydrolysis. Harper, N.J. (1962) in Jucker, ed. Progress in Drug Research, 4:221-294; Morozowich et al. (1977) in E. B. Roche ed. Design of Biopharmaceutical Properties through Prodrugs and Analogs, APhA (Acad. Pharm. Sci.); E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Curr. Pharm. Design. 5(4):265-287; Pauletti et al. (1997) Adv. Drug. Delivery Rev. 27:235-256; Mizen et al. (1998) Pharm. Biotech. 11:345-365; Gaignault et al. (1996) Pract. Med. Chem. 671-696; M. Asghamejad (2000) in G. L. Amidon, P. I. Lee and E. M. Topp, Eds., Transport Proc. Pharm. Sys., Marcell Dekker, p. 185-218; Balant et al. (1990) Eur. J. Drug Metab. Pharmacokinet., 15(2): 143-53; Balimane and Sinko (1999) Adv. Drug Deliv. Rev., 39(1-3):183-209; Browne (1997). Clin. Neuropharm. 20(1): 1-12; Bundgaard (1979) Arch. Pharm. Chemi. 86(1): 1-39; H. Bundgaard, ed. (1985) Design of Prodrugs, New York: Elsevier; Fleisher et al. (1996) Adv. Drug Delivery Rev, 19(2): 115-130; Fleisher et al. (1985) Methods Enzymol. 112: 360-81; Farquhar D, et al. (1983) J. Pharm. Sci., 72(3): 324-325; Han, H. K. et al. (2000) AAPS Pharm Sci., 2(1): E6; Sadzuka Y. (2000) Curr. Drug Metab., 1:31-48; D. M. Lambert (2000) Eur. J. Pharm. Sci., 11 Suppl 2:S1 5-27; Wang, W. et al. (1999) Curr. Pharm. Des., 5(4):265.

The imaging agent can also be provided as a lipid prodrug. Nonlimiting examples of U.S. patents that disclose suitable lipophilic substituents that can be covalently incorporated into the compound or in lipophilic preparations, include U.S. Pat. Nos. 5,149,794 (Sep. 22, 1992, Yatvin et al.); 5,194,654 (Mar. 16, 1993, Hostetler et al., 5,223,263 (Jun. 29, 1993, Hostetler et al.); 5,256,641 (Oct. 26, 1993, Yatvin et al.); 5,411,947 (May 2, 1995, Hostetler et al.); 5,463,092 (Oct. 31, 1995, Hostetler et al.); 5,543,389 (Aug. 6, 1996, Yatvin et al.); 5,543,390 (Aug. 6, 1996, Yatvin et al.); 5,543,391 (Aug. 6, 1996, Yatvin et al.); and 5,554,728 (Sep. 10, 1996; Basava et al.).

In one embodiment, pharmaceutical compositions including at least one compound of Formulas (I)-(V) or a labeled compound of Formula (VI)-(X) are provided. A host, including a human, suffering from, or at risk for, a disorder mediated by CXCR4 receptors can be diagnosed by administering an effective amount of a pharmaceutical composition of the active compound. Specifically, the diagnostics described herein can be used in a host, including a human, suffering from, or at risk of a proliferative or inflammatory disorder by administering an effective amount of a pharmaceutical composition of the imaging agent.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The compound or a pharmaceutically acceptable prodrug or salts thereof can also be mixed with other active materials that do not impair the desired action, or with materials that supplement the desired action, such as antibiotics, antifungals, anti-inflammatories, or antiviral compounds, or with additional chemotherapeutic agents. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation. If administered intravenously, preferred carriers are physiological saline or phosphate buffered saline (PBS).

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

### Kits

Kits comprising packaged imaging compositions described herein are provided. The imaging compositions for use in the kits comprise a radiolabeled CXCR4 antagonists and a pharmaceutically acceptable carrier. In certain embodiments, the packaged imaging composition includes indicia including at least one of: instructions for using the composition to image a host, or host samples (e.g., cells or tissues) for expression of CXCR4 receptors, which can be used as an indicator of conditions including, but not limited to, cancer, a tumor, cancer progression, angiogenesis, inflammation, and metastasis. In embodiments, the kit may include instructions for using the composition to assess therapeutic effect of a drug protocol administered to a patient, instructions for using the composition to selectively image malignant cells and tumors, and instructions for using the composition to predict metastatic potential.

This disclosure encompasses kits that include, but are not limited to, labeled CXCR4 antagonists, for example [¹⁸F]M508F, and directions (written instructions for their use). The components listed above can be tailored to the particular biological event to be monitored as described herein. The kit can further include appropriate buffers and reagents known in the art for administering various combinations of the components listed above to the host cell or host organism.

In certain preferred embodiments, the present disclosure provides a kit including from about 1 to 30 mCi of the radionuclide-labeled imaging agent described herein in combination with a pharmaceutically acceptable carrier. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

### Processes for the Preparation of CXCR4 Antagonist

### General Methods.

¹H NMR or ¹³C NMR spectra were recorded either on 400 MHz or 100 MHz NOVA Spectrometer or 600 MHz or 150 MHz NOVA Spectrometer. The spectra obtained were referenced to the residual solvent peak. They were recorded in deuterated chloroform, dimethyl sulfoxide-d6, deuterium oxide or acetone-d6. Melting points were taken on a Thomas Hoover capillary melting point apparatus and are uncorrected. Low-resolution EI mass spectra were recorded on a JEOL spectrometer. Element analyses were performed by Atlantic Microlab (Norcross, Ga.). Flash column chromatography was performed using Scientific Absorbent Incorporated Silica Gel 60. Analytical thin layer chromatography (TLC) was performed on precoated glass backed plates from Scientific Adsorbents Incorporated (Silica Gel 60 F₂₅₄). Plates were visualized using ultraviolet or iodine vapors or phosphomolybdic acid (PMA).

Method A: Reductive Amination Between Aldehydes/Ketones and Amines

(Abdel-Magid, et al. (1996) J. Org. Chem. 61:3849-3862). 1.0 eq. dialdehydes or ketones and 2.0 eq. amines were mixed in 1,2-dichloroethane and then treated with 3.0 eq. sodium triacetoxyborohydride (1.0-2.0 mol eq. acetic acid may also be added in reactions of ketones). The mixture was stirred at room temperature under an argon or nitrogen atmosphere for hours until the disappearance of the reactants in TLC plates. The reaction mixture was quenched by adding 1 N NaOH, and the product was extracted by ethyl ether, washed by Brine and dried by anhydrous MgSO₄. The solvent was evaporated to give the crude free base which could be purified by chromatography. The free base was dissolved in ethanolic hydrochloride or tartaric acid to give the salts which usually can recrystallize from MeOH/Et₂O.

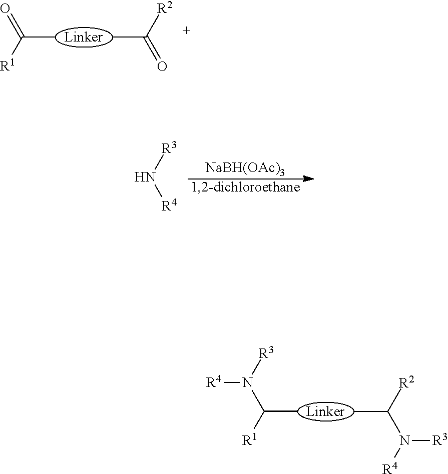

Method B: Reduction of Amides

(Micovic and Mihailovic (1953) J. Org. Chem. 18:1190). The amides could be prepared from the corresponding carboxylic acid or carboxylic chlorides. A mixture of carboxylic acid and thionyl chloride was refluxed for hours in an anhydrous system with a condenser equipped with a NaOH trap at the top. The excess thionyl chloride was removed under reduced pressure to get the carboxylic chloride. The carboxylic chloride was dissolved in dichloromethane following the addition of 2.0 eq. amine and 3 eq. pyridine. The mixture was stirred at room temperature until the disappearance of the reactants in the TLC plates. The solvent was removed under reduced pressure to get the crude amides which can be purified by chromatography.

The mixture of 1 eq. amide and 1.9 eq. LiAlH₄ in THF was refluxed until the disappearance of the amide from TLC plates. Then the solution was quenched with the addition of water and 15% NaOH aqueous as described in lit.5 and extracted with ethyl ether, dried over MgSO₄. Removal of the solvent gave the free amine product which can be purified by the chromatography. The free base dissolved in ethanolic hydrochloride or tartaric acid to give the salts which usually can recrystallize from MeOH/Et₂O.

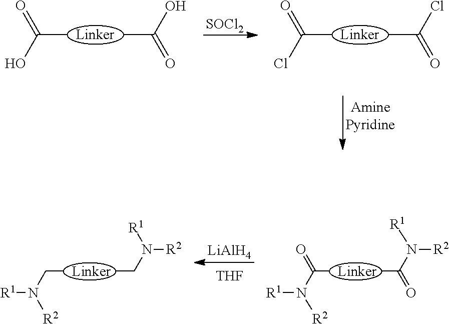

Method C: Nucleophilic Addition Between Amines and Chloropyrimidines.

1.0 eq. of diamine dihydrohalide, 5.0 eq. of trialkylamine base and 1.0 eq. of the appropriate chloropyrimidine in dimethylformamide were stirred together at elevated temperatures (80-130° C.) for hours. The reaction is diluted with aqueous saturated NaHCO₃. The aqueous phase is twice extracted with EtOAc. The combined organic phases are washed with Brine and dried by anhydrous MgSO₄. The solvent was evaporated and the resulting residue was purified by silica gel chromatography.

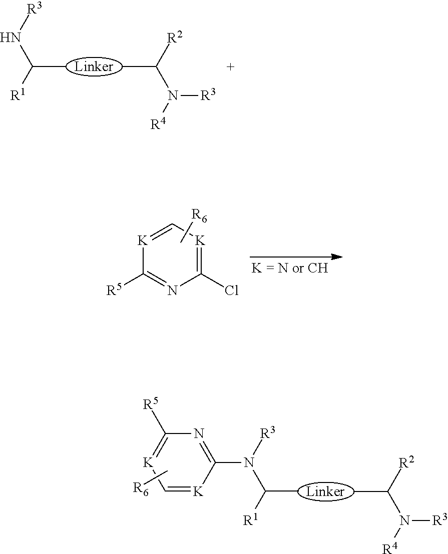

Method D: Removal of Tert-Butylcarbamate Protecting Groups to Provide Free Amines.

To a solution of 1.0 eq. of tert-butylcarbamate protected amine in methanol was added dropwise 10.0 eq. thionyl chloride. The reaction mixture was stirred at ambient temperature for 45 min before the solvent was evaporated provided the product as a crude product as a hydrochloride salt that is used without further purification.

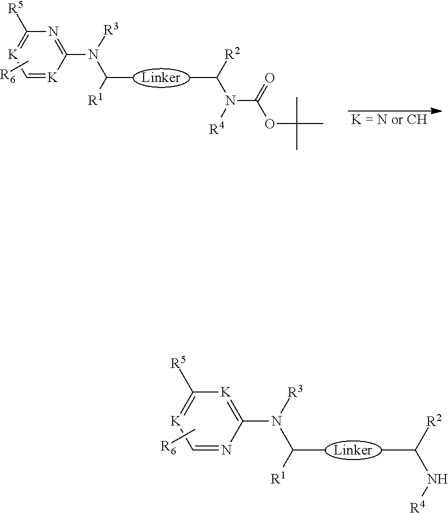

Method E: Nucleophilic Addition Between Amines and Chloropyrimidines.

1.0 eq. of amine hydrochloride, 5.0 eq. of trialkylamine base and 1.0 eq. of a 4-chloro-2-thiomethylpyrimidine in dimethylformamide were stirred together at elevated temperatures (80-130° C.) for hours. The reaction is diluted with aqueous saturated NH₄Cl. The aqueous phase is twice extracted with EtOAc. The combined organic phases are washed with Brine and aqueous saturated NaHCO₃ and dried by anhydrous MgSO₄. The solvent was evaporated and the resulting residue was purified by silica gel chromatography.

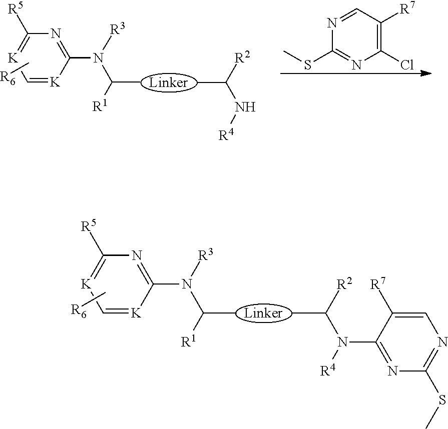

Method F: Oxidation of Thiomethylpyrimidine.

To a cold (0° C.) solution of 1.0 eq. thiomethylpyrimidine in CH₂Cl₂ was added 1.5 eq. 3-chloroperoxybenzoic acid (Oxone may also be used as an oxidant for this reaction). The mixture was stirred at 0° C. for hours until the disappearance of the reactants in TLC plates. The reaction was quenched by pouring the reaction mixture into aqueous saturated NaHCO₃, and the product was extracted by CH₂Cl₂. The organic phase was dried with anhydrous MgSO₄ to provide a mixture of sulfoxide and sulfone product mixture that is pure enough to be used without further purification.

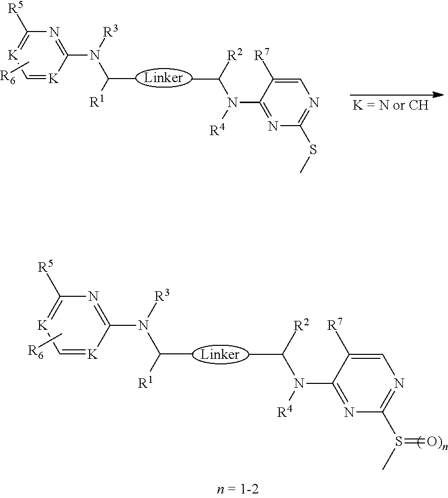

Method G: Displacement of Sulfoxide/Sulfone Functionalities.

A mixture of 1.0 eq. sulfoxide/sulfone mixture, 10.0 eq. of amine and 2.0 eq. of trialkylamine in dioxane were stirred together at reflux for hours. The reaction is diluted with aqueous saturated NaHCO₃. The aqueous phase is twice extracted with EtOAc. The combined organic phases are dried by anhydrous MgSO₄. The solvent was evaporated and the resulting residue was purified by silica gel chromatography.

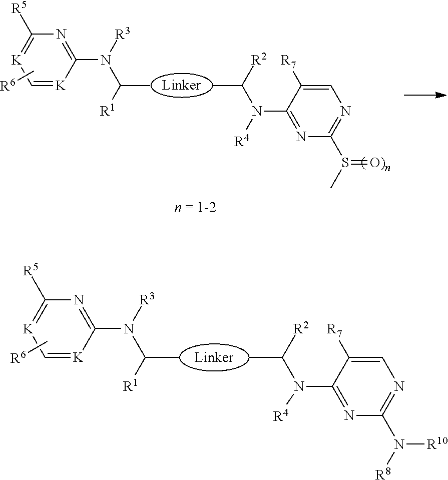

Method H: Acylation of Amines for Generation of Pyrimidine Carboxamides.

A mixture of 1.0 eq. the appropriate pyrimidine acid chloride and 1.0 eq. of an amine in tetrahydrofuran were stirred together at ambient temperature (80-130° C.) for several hours. The reaction is diluted with water. The aqueous phase is extracted with EtOAc. The organic phase is twice washed with water, twice washed with Brine, and dried by anhydrous sodium sulfate. The solvent was evaporated and the resulting residue was used without further purification.

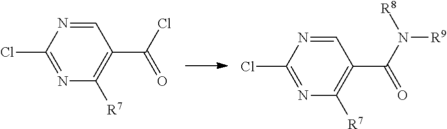

Method I: Displacement of 5-acyl-2-chloropyrimidines.

A mixture of 1.0 eq. (aminomethyl)benzyl pyrimidine, 1.0 eq. of the appropriate 2-chloropyrimidine and 5.0 eq. of trialkylamine in dimethylformamide were stirred together at elevated temperatures (80-130° C.) for several hours. The reaction is diluted with water. The aqueous phase is twice extracted with EtOAc. The combined organic phases are twice washed with water, twice washed with Brine, and dried by anhydrous sodium sulfate. The solvent was evaporated and the resulting residue was purified by silica gel chromatography.

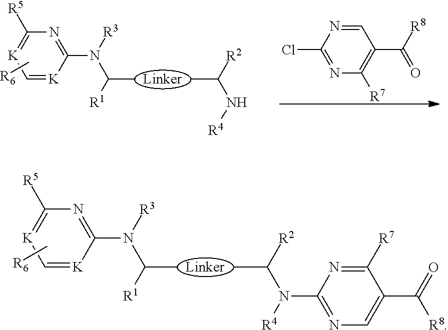

Radiofluorination Methods:

Generally, radiofluorination reactions are nucleophilic substitutions in homoaromatic, heteroaromatic, and aliphatic series (see, for example, Kilbourn M R. In: Kilbourn M R Ed. Nuclear Science Series Fluorine-18 labeling of radiopharmaceuticals. Washington, National Academy Press: 1990; 1-149; Lasne M-C, et al. In: Krause W Ed. Topics in current chemistry—Chemistry of β+ emitting compounds based on fluorine-18. Berlin Heidelberg, Springer-Verlag 2002; Vol. 222: 201-258; Done F. Preparation of [18F]-labeled fluoropyridines: Advances in radiopharmaceutical design. Curr Pharm Design 2005; 11: 3221-3235). Homoaromatic nucleophilic substitutions with fluoride usually require activated aromatic rings, bearing both a good leaving group (e.g. a halogen, a nitro- or a trimethylammonium group) and a strong electron-withdrawing substituent (e.g. a nitro-, cyano- or acyl group) preferably placed para to the leaving group, whereas aliphatic nucleophilic substitutions only require a good leaving group (usually a halogen or a sulphonic

acid derivative such as mesylate, tosylate, or triflate). Labelling procedures involve pre-activation of cyclotron produced, no-carrier-added, aqueous [¹⁸F]fluoride by evaporation to dryness from an added base (for example K₂CO₃) and, for example, the added kryptand Kryptofix-222, in order to form the fluoride anion as a K[¹⁸F]F-K222 complex (Hamacher K, et al. Efficient stereospecific synthesis of no-carrier-added 2-[¹⁸F]-fluoro-2-deoxy-D-glucose using aminopolyether supported nucleophilic substitution. J Nucl Med Biol 1986; 27: 235-8.). Nucleophilic substitutions are then performed in an aprotic polar solvent under alkaline conditions, either directly on a suitable and generally complex precursor of the target molecule or on a small labelled precursor followed by a multi-step indirect synthetic approach.

Nucleophilic heteroaromatic substitution at the ortho-position of pyridinyl moerty with no-carrrier-added [¹⁸F]fluoride as its activated K[¹⁸F]F-K222 complex is the most efficient method for the radiosynthesis of radiopharmaceuticals of high specific radioactivity when compared to both homoaromatic and aliphatic nuleophilic radiofluorination. The CXCR4 antagonist compounds described herein, in particular, those comprising fluoropyridinyl moieties (WZ95) or fluoropyrimidinyl moieties, can be successfully labeled by [¹⁸F] by nucleophilic substitution. The radiochemical yield of ¹⁸F-M508F is more than 30% which is can be purified by HPLC with more than 90% purity.

The CXCR4 antagonist compounds described herein could also be labeled by radioisotope bromine or iodine through traditional labeling procedures such as tributyltin derivatives. (See, for example, Plisson C, et al. Synthesis and in vivo evaluation of fluorine-18 and iodine-123 labeled 2beta-carbo(2-fluoroethoxy)-3beta-(4′-((Z)-2 iodoethenyl)phenyl)nortropane as a candidate serotonin transporter imaging agent. J Med Chem 2007; 50(19):4553-60; Plisson C, et al. Synthesis, radiosynthesis, and biological evaluation of carbon-11 and iodine-123 labeled 2beta-carbomethoxy-3beta-[4′-((Z)-2-haloethenyl)phenyl]tropanes: candidate radioligands for in vivo imaging of the serotonin transporter. J Med Chem 2004; 47(5):1122-35; Li Z, et al. Synthesis of structurally identical fluorine-18 and iodine isotope labeling compounds for comparative imaging. Bioconjug Chem 2003; 14(2):287-94; Goodman M. M., et al. Synthesis and characterization of iodine-123 labeled 2beta-carbomethoxy-3beta-(4′-((Z)-2-iodoethenyl)phenyl)nortropane: A ligand for in vivo imaging of serotonin transporters by single-photon-emission tomography. J Med Chem 2003; 46(6):925-35; Maziere B, et al. ⁷⁶Br-beta-CBT, a PET tracer for investigating dopamine neuronal uptake. Nucl Med Biol 1995; 22(8):993-7).

### MRS and MRI Labeling Methods:

The CXCR4 antagonist compounds described herein may be labeled to prepare MRS or MRI suitable imaging agents for example by incorporation of ¹³C, which may be accomplished by general organic chemistry techniques known to the art, see for example J. March, Advanced Organic Chemistry: Reactions. Mechanisms and Structure (3^{rd} Edition, 1985), the contents of which are hereby incorporated by reference.

### Optical Imaging Labeling Methods:

The CXCR4 antagonist compounds described herein may be labeled to prepare imaging agents suitable for optical imaging by labeling the compound with a dye. For example, certain CXCR4 antagonist compounds comprise secondary or primary amine groups can be linked to a dye compound as shown in Scheme 1. Other CXCR4 antagonist compounds comprise free sulfhydryl (—SH)) groups which can be linked to a dye compound through Maleimide chemistry as shown in Scheme 2.

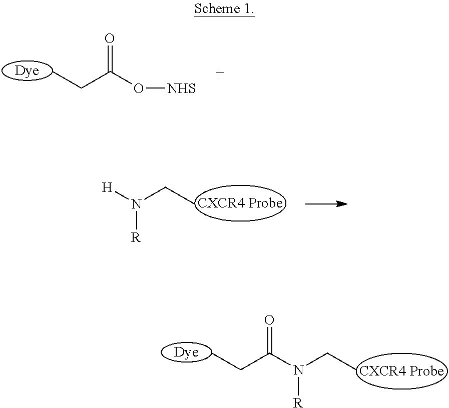

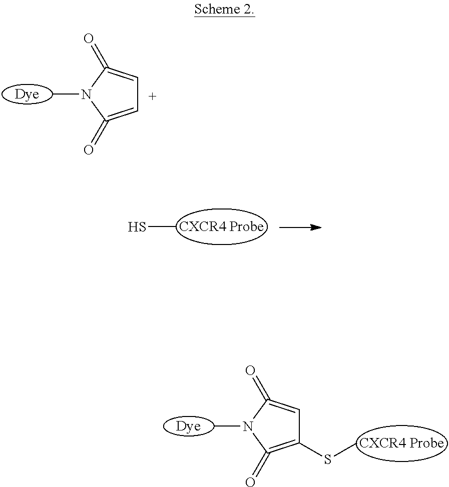

### Stereoisomerism and Polymorphism

Compounds of the present invention having a chiral center may exist in and be isolated in optically active and racemic forms. The present invention encompasses any racemic, optically-active, diastereomeric, polymorphic, or stereoisomeric form, or mixtures thereof, of a compound of the invention, which possess the useful properties described herein.

Examples of methods to obtain optically active materials are known in the art, and include at least the following.
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chrial catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xi) xii) extraction with chiral solvents—a technique whereby the enantiomers are separated by virtue of preferential dissolution of one enantiomer into a particular chiral solvent;
xii) xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

### Diseases

The imaging agents, compositions and methods described herein are particularly useful for the imaging or detection of a disorder associated with CXCR4 receptor binding or activation, and particularly a proliferative disorder, including cancer metastasis. However, numerous other diseases have been associated with CXCR4 receptor signaling.

Cancer is a general term for diseases in which abnormal cells divide without control. Cancer cells can invade nearby tissues and can spread through the bloodstream and lymphatic system to other parts of the body. It has been discovered that the expression of CXCR4 receptors by cancer cells is a strong indicator of the metastatic potential of such cells. It has also been demonstrated that the administration of a CXCR4 antagonist to a host, for example a mammal, inhibits or reduces the metastasis of tumor cells, in particular breast cancer and prostate cancer.

There are several main types of cancer, and the disclosed compositions can be used to treat any type of cancer. For example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. A solid tumor is an abnormal mass of tissue that usually does not contain cysts or liquid areas. A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. The compositions described herein can be used to image, detect, and follow the progression of tumor cells and their metastases, and thereby assist in the diagnosis and treatment of the cancer.

Representative cancers that may treated with the disclosed compositions and methods include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others (for a review of such disorders, see Fishman et al., 1985, Medicine, 2d Ed., J. B. Lippincott Co., Philadelphia and Murphy et al., 1997, Informed Decisions The Complete Book of Cancer Diagnosis, Treatment, and Recovery, Viking Penguin, Penguin Books U.S.A., inc., United States of America).

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth) and continue their rapid growth and division in this new location. This is called metastasis. Once malignant cells have metastasized, achieving cure is more difficult. CXCR4 receptor antagonists are shown herein to be useful for the detection and prediction of metastasis of cancer cells.

Benign tumors have less of a tendency to invade and are less likely to metastasize. They do divide in an uncontrolled manner, though. Depending on their location, they can be just as life threatening as malignant lesions. An example of this would be a benign tumor in the brain, which can grow and occupy space within the skull, leading to increased pressure on the brain. Since CXCR4 is also produced to some extent by all tumors, but to a much greater extent by metastatic tumors, the compositions provided herein can be used to differentiate malignant and benign tumors.

Recently, an animal model of bone metastasis was generated by the intercardiac injection of MDA-MB-231 cells into female SCID mice (Kang, et al., (2003) Cancer Cell. 3(6):53749). A subsequent microarray analysis on a sub-population of MDA-MB-231 cells with elevated metastatic activity isolated from the mouse showed that one of the six genes responsible for the metastatic phenotype was CXCR4. Over-expression of CXCR4 alone in original MDA-MB-231 cells significantly increased the metastatic activity of the cells. In samples collected from various breast cancer patients, Muller et al. found that the level of expression of CXCR4 is higher in primary tumors relative to normal mammary gland or mammary epithelial cells (Muller et al, (2001) Nature 410(6824):50-6). By contrast, CXCL12 is highly expressed in the most common destinations of breast cancer metastasis including the lymph nodes, lung, liver, and bone marrow. Current evidence suggests that the expression of CXCR4 on breast cancer cell surfaces may direct such cells to the sites that are known to express high levels of CXCL12. It has been shown that CXCR4 antibody treatment inhibits metastasis to regional lymph nodes while all isotype controls metastasized to the lymph nodes and lungs (Muller et al, (2001) Nature 410(6824):50-6). These data indicate that neutralization of the interaction between CXCR4 and its ligand, CXCL12, by a CXCR4 antibody can significantly impair metastasis of breast cancer cells to the lymph nodes and lungs. Taken together with the data provided in the examples below, CXCR4 and CXCL12 appear to play critical roles in breast cancer and head and neck cancer metastasis, thus, detection and quantification of CXCR4 expression levels can help in the detection of cancer and metastasis, the prediction of metastasis, as well as in monitoring the progression of cancer and/or the effectiveness of treatment regimens.

The imaging agents, compositions and methods described herein can be used to image or detect disorders of abnormal cell proliferation generally, examples of which include, but are not limited to, types of cancers and proliferative disorders listed below. Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. In normal skin the time required for a cell to move from the basal layer to the upper granular layer is about five weeks. In psoriasis, this time is only 6 to 9 days, partially due to an increase in the number of proliferating cells and an increase in the proportion of cells which are dividing (G. Grove, Int. J. Dermatol. 18:111, 1979). Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue can cause, for example, restenosis, retinopathies and atherosclerosis. The advanced lesions of atherosclerosis result from an excessive inflammatory-proliferative response to an insult to the endothelium and smooth muscle of the artery wall (Ross, R. Nature 1993, 362:801-809). Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells (See, e.g., Harris, E. D., Jr. (1990) The New England Journal of Medicine, 322:1277-1289), and to be caused by autoantibodies produced against collagen and IgE.

Other disorders that can include an abnormal cellular proliferative component include Behcet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

Examples of proliferative disorders which can be the primary tumor that is imaged or detected, or which can be the site from which metastasis derives, include but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvis, skin, soft tissue, spleen, thorax, and urogenital tract.

Specific types of diseases include Acute Childhood Lymphoblastic Leukemia; Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphorria, Adult Lymphocytic Leukemia, Adult Non-Hodgkin's Lymphoma, Adult Primary Liver Cancer, Adult Soft Tissue Sarcoma, AIDS-Related Lymphorria, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphorria, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalanic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma. Endometrial Cancer, Ependymoma, Epithelial Cancer, Esophageal Cancer, Ewing's Sarcoma and Related Tumors, Exocrine Pancreatic Cancer, Extraeranial Germ Cell Tumor, Extragonadal Germ Cell Tumor, Extrahepatie Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lympho proliferative Disorders, Macroglobulinemia, Male Breast Cancer, Malignant Mesothelioma, Malignant Thymoma, Medulloblastomia, Melanoma, Mesothelioma, Metastatie Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyrigeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid, Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethial Cancer, Uterine Cancer, Uterine Sarcoma, Vaginal Cancer, Visual Pathway and Hypothalarruc Glioma, Vulvar Cancer, Waldenstroin's Macroglobulinemia, Wilm's Tumor, and any other hyperproliferative disease located in an organ system listed above.

Hyperplastic disorders include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, foca epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia; leukemia (including acute leukemia (e.g., acute lymphocytic leukemia, acute myelocytic leukemia (including myeloblastic, promyelocytic, mylomonocytic, monocytic, and erythroleukemia)) and chronic leukemia (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, Sarcomas and, carcinomas such as fibrosarcoma, myxosarcoma, fiposarcoma, chondrosarcoma, osteogenic sarcoma, chordoma, anglosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendrogliomia, menangioma, melanoma, neuroblastoma, and retinoblastoma.

In a separate embodiment, a method for the imaging of age-related macular degeneration (ARMD) and other pathogenic states involving macular retinal pigment epithelial (RPE) cells including administering at least one compound described herein is provided.

CXCR4 plays a crucial role in ocular diseases involving the retina such as age-related macular degeneration (ARMD). The retinal pigment epithelium has a major role in the physiological renewal of photoreceptor outer segments in the provision of a transport and storage system for nutrients essential to the photoreceptor layer. The retinal pigment epithelial (RPE) cells predominantly express CXCR4 receptors. (Crane, et al. (2000) J. Immunol. 165: 4372-4278). CXCR4 receptor expression on human retinal pigment epithelial cells from the blood-retina barrier leads to chemokine secretion and migration in response to stromal cell-derived factor 1a. J. Immunol. 200; 165: 4372-4278). The level of CXCR4 mRNA expression increases upon stimulation with IL-1β or TNFα (Dwinell, et al. (1999) Gastroenterology. 117: 359-367). RPE cells also migrated in response to CXCL12 indicating that CXCL12/CXCR4 interactions may modulate the effects of chronic inflammation and subretinal neovascularization at the RPE site of the blood-retina barrier. (Crane I J, Wallace C A, McKillop-Smith S, Forrester J V. CXCR4 receptor expression on human retinal pigment epithelial cells from the blood-retina barrier leads to chemokine secretion and migration in response to stromal cell-derived factor 1a. J. Immunol. 200; 165: 4372-4278).

Age-related macular degeneration is characterized by both primary and secondary damage of macular RPE cells. Early stages of ARMD are characterized by macular drusen, and irregular proliferation and atrophy of the RPE. The late stages of ARMD present with geographic RPE atrophy, RPE detachment and rupture, choroidal neovascularaization and fibrovascular disciform scarring. Common first symptoms include metamorphopisia and/or general central vision loss resulting in reading disability and difficulties in detecting faces. Late stages of ARMD cause central scomota, which is extremely disabling if occurrence is bilateral (Bressler and Bressler (1995) Ophthalmology. 1995; 102: 1206-1211).

In a separate embodiment, a method for the treatment of, prevention of, or reduced severity of inflammatory disease states, neovascularization, and wound healing including administering at least one compound described herein is provided.

Vascular endothelial cells express a multitude of chemokine receptors, with CXCR4 being particularly prominent (Gupta, et al. (1998) J Biol. Chem. 273: 4282; Volin, et al. (1998) Biochem Biophys Res Commnun. 242: 46).

A RT-PCR based strategy which utilized CXCR4 specific primers demonstrated that mRNA for the chemokine receptor CXCR4 is expressed not only in primary cultures and transformed type II alveolar epithelial cells (pneumocytes) but also in a number of epithelial cell lines derived from various other tissues. (Murdoch, et al. (1998) Immunology. 98(1): 36-41). Whether CXCR4 participates in inflammatory responses remains unclear. CXCR4 expressed on the epithelium may facilitate the recruitment of immune cells to sites of inflammation by direct effects on epithelial cells. CXCR4 may also have other functional roles within the immune response or participate in wound healing or neovascularization. CXCR4 may also be involved in the pathophysiology of several acute or chronic inflammatory disease states associated with the epithelium. (Murdoch, et al. (1999) Immunology. 98(1): 36-41).

Certain inflammatory chemokines can be induced during an immune response to promote cells of the immune system to a site of infection. Inflammatory chemokines function mainly as chemoattractants for leukocytes, recruiting monocytes, neutrophils and other effector cells from the blood to sites of infection or tissue damage. Certain inflammatory chemokines activate cells to initiate an immune response or promote wound healing. Responses to chemokines include increasing or decreasing expression of membrane proteins, proliferation, and secretion of effector molecules.

In a particular embodiment, the compounds of the invention can be administered to a host at risk of, or suffering from, an inflammatory condition. In one embodiment, the compounds are administered for the treatment or prophylaxis of an inflammatory disorder. In certain embodiments, the inflammatory disorder or condition is mediated by chemokines.

Generally, inflammatory disorders include, but are not limited to, respiratory disorders (including asthma, COPD, chronic bronchitis and cystic fibrosis); cardiovascular related disorders (including atherosclerosis, post-angioplasty, restenosis, coronary artery diseases and angina); inflammatory diseases of the joints (including rheumatoid and osteoarthritis); skin disorders (including dermatitis, eczematous dermatitis and psoriasis); post transplantation late and chronic solid organ rejection; multiple sclerosis; autoimmune conditions (including systemic lupus erythematosus, dermatomyositis, polymyositis, Sjogren's syndrome, polymyalgia rheumatica, temporal arteritis, Behcet's disease, Guillain Barré, Wegener's granulomatosus, polyarteritis nodosa); inflammatory neuropathies (including inflammatory polyneuropathies); vasculitis (including Churg-Strauss syndrome, Takayasu's arteritis); inflammatory disorders of adipose tissue; and proliferative disorders (including Kaposi's sarcoma and other proliferative disorders of smooth muscle cells).

In one embodiment, compounds, compositions and methods of treatment of respiratory disorders comprising administering a compound are provided wherein the compound is as described herein. Respiratory disorders that may be prevented or treated include a disease or disorder of the respiratory system that can affect any part of the respiratory tract. Respiratory disorders include, but are not limited to, a cold virus, bronchitis, pneumonia, tuberculosis, irritation of the lung tissue, hay fever and other respiratory allergies, asthma, bronchitis, simple and mucopurulent chronic bronchitis, unspecified chronic bronchitis (including chronic bronchitis NOS, chronic tracheitis and chronic tracheobronchitis), emphysema, other chronic obstructive pulmonary disease, asthma, status asthmaticus and bronchiectasis. Other respiratory disorders include allergic and non-allergic rhinitis as well as non-malignant proliferative and/or inflammatory disease of the airway passages and lungs. Non-malignant proliferative and/or inflammatory diseases of the airway passages or lungs means one or more of (1) alveolitis, such as extrinsic allergic alveolitis, and drug toxicity such as caused by, e.g. cytotoxic and/or alkylating agents; (2) vasculitis such as Wegener's granulomatosis, allergic granulomatosis, pulmonary hemangiomatosis and idiopathic pulmonary fibrosis, chronic eosinophilic pneumonia, eosinophilic granuloma and sarcoidoses.

In one embodiment, the compounds of the invention are administered to a patient suffering from a cardiovascular disorder related to inflammation. Cardiovascular inflammatory disorders include atherosclerosis, post-angioplasty, restenosis, coronary artery diseases, angina, and other cardiovascular diseases.

In certain embodiments the disorder is a non-cardiovascular inflammatory disorder such as rheumatoid and osteoarthritis, dermatitis, psoriasis, cystic fibrosis, post transplantation late and chronic solid organ rejection, eczematous dermatitis, Kaposi's sarcoma, or multiple sclerosis. In yet another embodiment, the compounds disclosed herein can be selected to treat anti-inflammatory conditions that are mediated by mononuclear leucocytes.

In addition, the invention is directed to methods of treating animal subjects, in particular, veterinary and human subjects, to enhance or elevate the number of progenitor cells and/or stem cells. The progenitor and/or stem cells may be harvested and used in cell transplantation. In one embodiment, bone marrow progenitor and/or stem cells are mobilized for myocardial repair. Further, the invention is directed to methods of treating animal subjects, in particular, veterinary and human patients, who are defective in white blood cell (WBQ 8 count, or who would benefit from elevation of WBC levels using the compounds disclosed herein. Moreover, the invention is directed to methods of effecting regeneration of cardiac tissue in a subject in need of such regeneration using the disclosed compounds.

The compounds of the invention may be used for the treatment of diseases that are associated with immunosuppression such as individuals undergoing chemotherapy, radiation therapy, enhanced wound healing and bum treatment, therapy for autoinimune disease or other drug therapy (e.g., corticosteroid therapy) or combination of conventional drugs used in the treatment of autoinimune diseases and graft/transplantation rejection, which causes immunosuppression; immunosuppression due to congenital deficiency in receptor function or other causes; and infectious diseases, such as parasitic diseases, including but not limited to helminth infections, such as nematodes (round invention thus targets a broad spectrum of conditions for which elevation of progenitor cells and/or stem cells in a subject would be beneficial or, where harvesting of progenitor cells and/or stem cell for subsequent stem cell transplantation would be beneficial. In addition, the method of the invention targets a broad spectrum of conditions characterized by a deficiency in white blood cell count, or which would benefit from elevation of said WBC count.

The term “progenitor cells” refers to cells that, in response to certain stimuli, can form differentiated hematopoietic or myeloid cells. The presence of progenitor cells can be assessed by the ability of the cells in a sample to form colony-forming units of various types, including, for example, CFU-GM (colony-forming units, granulocytemacrophage); CFU-GEMM (colony-forming units, multipotential); BFU-E (burst-forming units, erythroid); HPP-CFC (high proliferative potential colony-forming cells); or other types of differentiated colonies which can be obtained in culture using known protocols. “Stem” cells are less differentiated forms of progenitor cells. Typically, such cells are often positive for CD34. Some stem cells do not contain this marker, however. In general, CD34+ cells are present only in low levels in the blood, but are present in large numbers in bone marrow.

The compounds of the invention may be administered as sole active ingredients, as mixtures of various compounds of Formula (I)-(V), and/or in admixture with additional active ingredients that are therapeutically or nutritionally useful, such as antibiotics, vitamins, herbal extracts, anti-inflammatories, glucose, antipyretics, analgesics, granulocyte-macrophage colony stimulating factor (GM-CSF), Interleukin-1 (IL-1), Interleukin-3 (IL-3), Interleukin-8 (IL-8), PIXY-321 (GM-CSF/IL-3 fusion protein), macrophage inflammatory protein, stem cell factor, thrombopoictin, growth related oncogene or chemotherapy and the like. In addition, the compounds of the invention may be administered in admixture with additional active ingredients that are therapeutically or nutritionally useful, such as antibiotics, vitamins, herbal extracts, anti-inflammatories, glucose, antipyretics, analgesics, and the like.

The binding of CXCL12 to CXCR4 has also been implicated in the pathogenesis of atherosclerosis (Abi-Younes et al. Circ. Res. 86, 131-138 (2000)), renal allograft rejection (Eitner et al. Transplantation 66, 1551-1557 (1998)), asthma and allergic airway inflammation (Yssel et al. Clinical and Experimental AllerD; 28, 104-109 (1998); J 1777771unol. 164, 59355943 (2000); Gonzalo et al. J limmunol. 165, 499-508 (2000)), Alzheimer's disease (Xia et al. J. Neurovirologv 5, 32-41 (1999)) and Arthritis (Nanlci et al. J. Immunol. 164, 5010-5014 (2000)).

The following examples describe specific aspects of the invention to illustrate the invention and provide a description of the present methods for those skilled in the art. The Examples should not be construed as limiting the invention as the examples merely provide specific methodology useful in the understanding and practice of the invention and its various aspects.

### EXAMPLES

### Example 1

### Preparation of N4-(4((2-¹⁸-fluoro,5-fluoropyrimidin-4-ylamino)methyl)benzyl)-N2-(2-morpholinoethyl)pyrimidine-2,4-diamine ([¹⁸F]M508F).

¹⁸F-M508F was prepared from N4-(4-((2-chloro-5-fluoropyrimidin-4-ylamino)methyl)benzyl)-N2-(2-morpholinoethyl)pyrimidine-2,4-diamine (M508Cl). The preparation of M508Cl is shown in Scheme 3. See US Published Application Number 2009-0099194 hereby incorporated by reference.

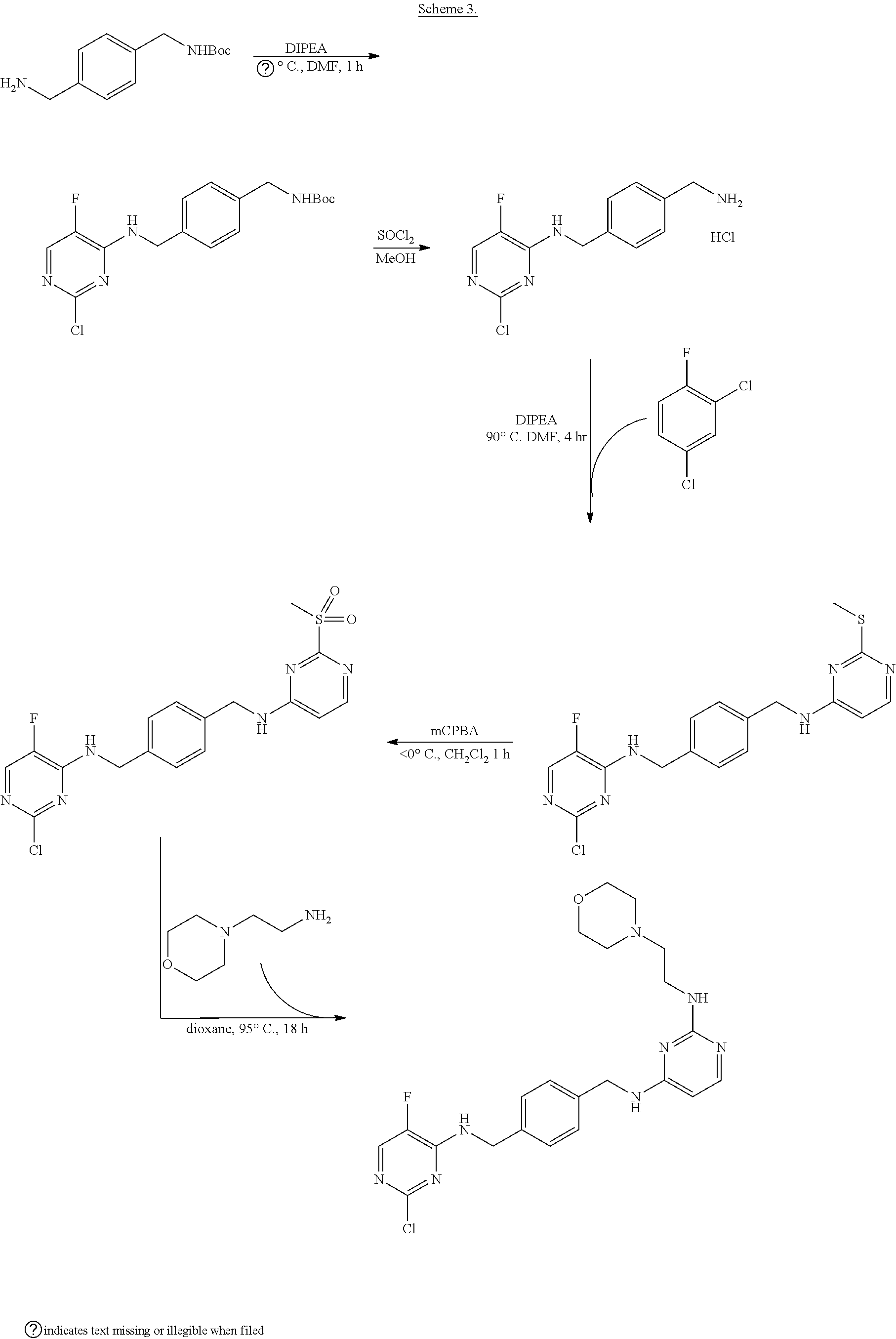

The substitution of the chloride with an 18-fluoride ligand was accomplished in one step, as shown in Scheme 4.

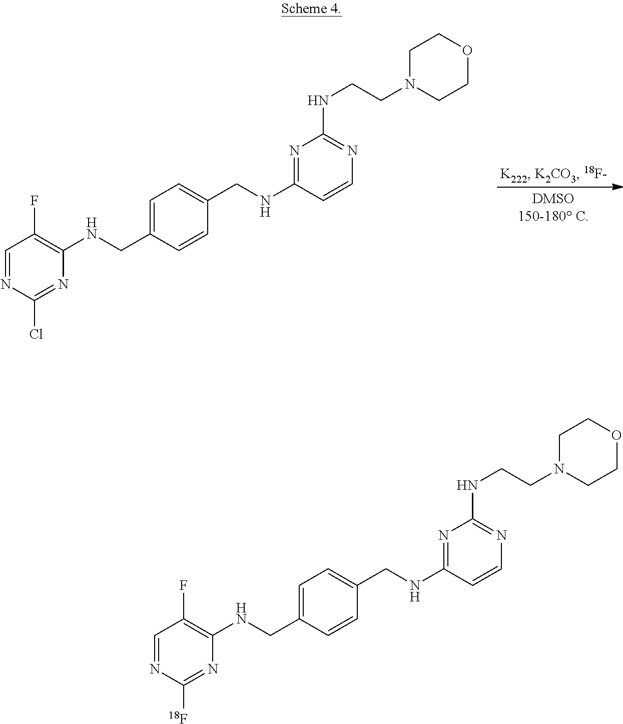

Fluorine-18 fluoride is prepared by the ¹⁸O (p,n) ¹⁸F reaction using ¹⁸O enriched water by PetNet Solutions at Yerkes National Primate Center. The aqueous solution is transferred into a vial located in a hot cell in the Yerkes Radiochemistry Laboratory. The solution is then transferred into a mini cell which houses a chemical process control unit where ¹⁸F— is trapped on an anion exchange resin cartridge. ¹⁸F— is then eluted with a potassium carbonate solution into a vessel containing Kryptofix 2,2,2 and the mixture is dried by azeotropic distillation with acetonitrile. ¹⁸F— is reacted with the M508Cl in DMSO at 170° C. to produce the final radioactive compound ¹⁸F-M508F, which is isolated from the fractions by C¹⁸ solid phase extraction and eluted by ethanol into a vial containing isotonic saline. The ¹⁸F-M508F saline solution is sterilized by filtration through a 0.2 micron filter for further study.

Alternative non-radioactive halogenated derivatives may be prepared using schemes above provided for M508Cl. For example, to prepare the non-radioactive F508M, N⁴-(4-((2-fluoro, 5-fluoropyrimidin-4-ylamino)methyl)benzyl)-N²-(2-morpholinoethyl)pyrimidine-2,4-diamine, one uses 2,4,5-trifluoropyrimidine, whereas M508Cl used 2,4-dichloro-5-fluoropyrimidine at the first step. For N⁴-(4-((2-bromo,5-fluoropyrimidin-4-ylamino)methyl)benzyl)-N²-(2-morpholinoethyl)pyrimidine-2,4-diamine one uses 2,4-dibromo-5-fluoropyrimidine.

### Example 2

### CXCR4 Binding Affinity of [18F]M508F

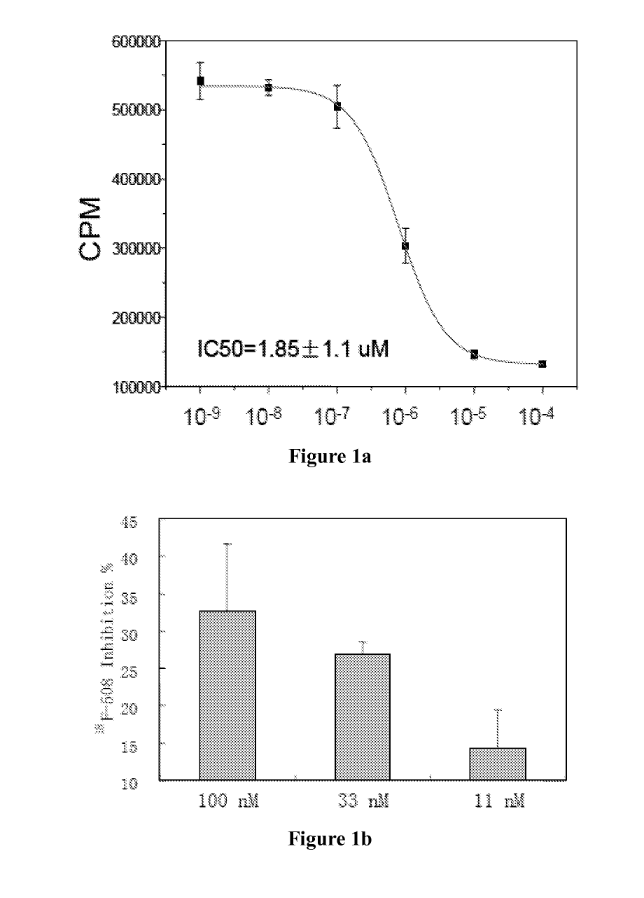

To determine whether [¹⁸F]M508F specifically and quantitatively would bind to CXCR4 on the cell surface, we tested this using the metastatic 686LN subclones that are CXCR4-positive in vitro. As discussed herein, Jacobson et al. labeled AMD3100 with ⁶⁴Cu (t_{1/2}=12.7 h) to produce ⁶⁴Cu-AMD3100; its binding affinity to CXCR4 was found to be 62.7 μM. Similarly, a competition assay was used to determine the binding affinity of [¹⁸F]M508F to CXCR4. Metastatic 686LN cells, incubated with a constant amount of [¹⁸F]-M508F and increasing concentrations of nonradioactive [¹⁹F]-M508F for 1 h at ambient temperature, were used in the competition binding assay. The binding affinity of [¹⁸F]M508F for CXCR4 was determined to be 1.85 μM, which was over 50 times better than ⁶⁴Cu-AMD3100 (FIG. 1a).

To determine whether it would bind to CXCL12-binding sites on CXCR4, we tested whether preincubation with CXCL12 for 15 mins before adding [¹⁸F]M508F blocked its binding to CXCR4 on cell surface. The competition binding assay used metastatic 686LN cells. The cells were incubated with a constant amount of [¹⁸F]-M508F and increasing concentrations of CXCL12 (ligand for CXCR4) for 1 h at RT. It was determined that CXCL12 blocked [¹⁸F]M508F binding to CXCR4-positive cells in a dose-dependent manner (FIG. 1b). The results of the competition assays support that [¹⁸F]-M508F is a CXCR4-targeting agent.

### Example 3

### Determining Tissue Biodistribution of the ¹⁸F-Labeled Radioligands in Mice by Tissue Harvesting

To determine the tissue biodistribution of [¹⁸F]M508F using a gamma counter in regular C57Black mice, we harvested animal organs, including blood, at 15 min, 30 min, 60 min, and 120 min post-injection and measured the uptake of radioactivity in various organs [radioactivity (% injected dose)/g] with n=5 per group. Biodistribution of our CXCR4-PET tracer showed fast blood and muscle clearance and accumulation in CXCR4-expressing

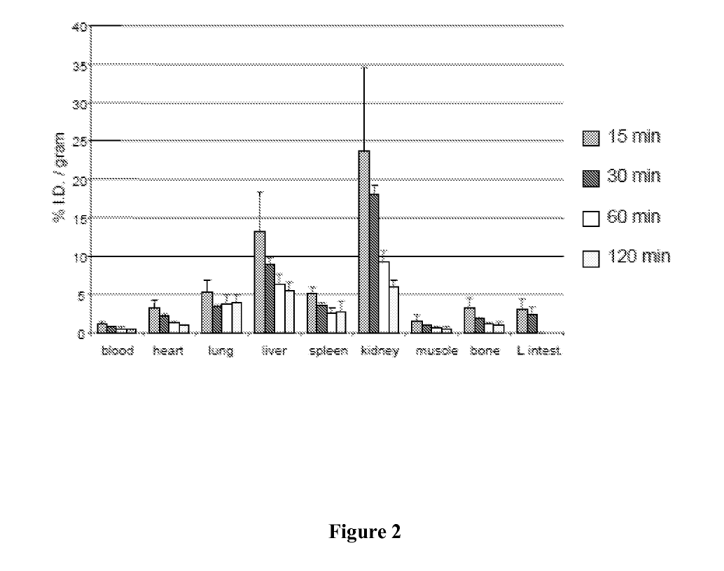

organs and tissues, a renal clearance pathway, and an anomalous specific accumulation in the liver. Results are shown in FIG. 2 as average vales +/−SE.

### Example 4

### MicroPET Imaging Study of [¹⁸F]M1508F in an Orthoscopic SCCHN Animal Model

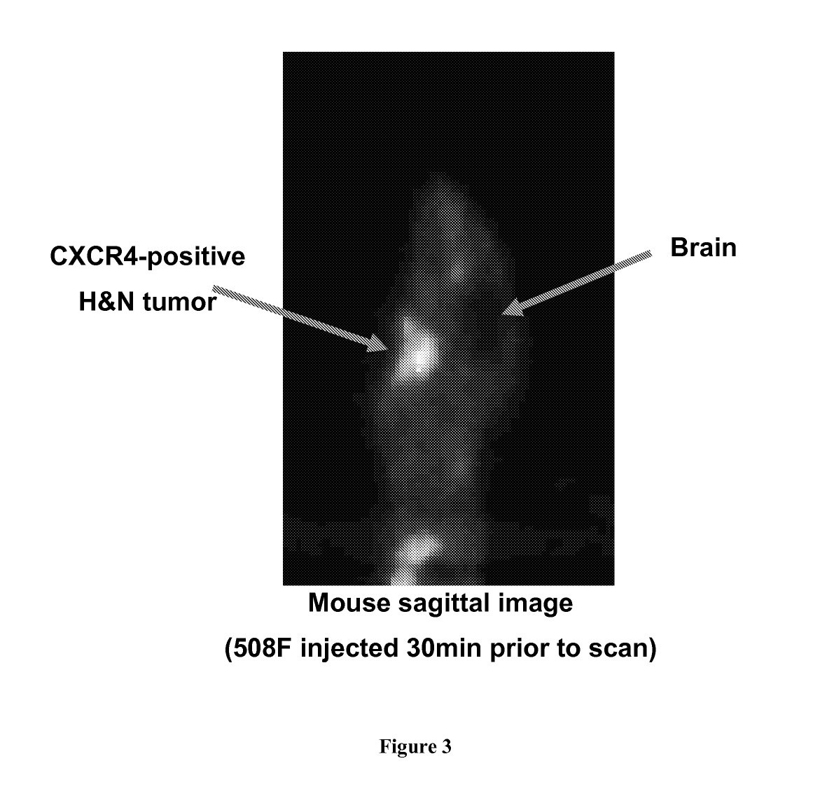

In vivo biodistribution and tumor uptake studies were performed to determine whether [¹⁸F]M508F can be used to image CXCR4-positive tumors in vivo. [¹⁸F]M508F-PET sagittal imaging of a mouse bearing CXCR4-positive orthothopic SCCHN tumors exhibited significant uptake of our CXCR4-PET tracer at 30 mins post-injection (FIG. 3). Of note, this image demonstrates that the [¹⁸F]M508F CXCR4-PET tracer does not cross the blood-brain-barrier.

### Example 5

### MicroPET/CT Imaging Study of [¹⁸F]M508F in an Edema Animal Model

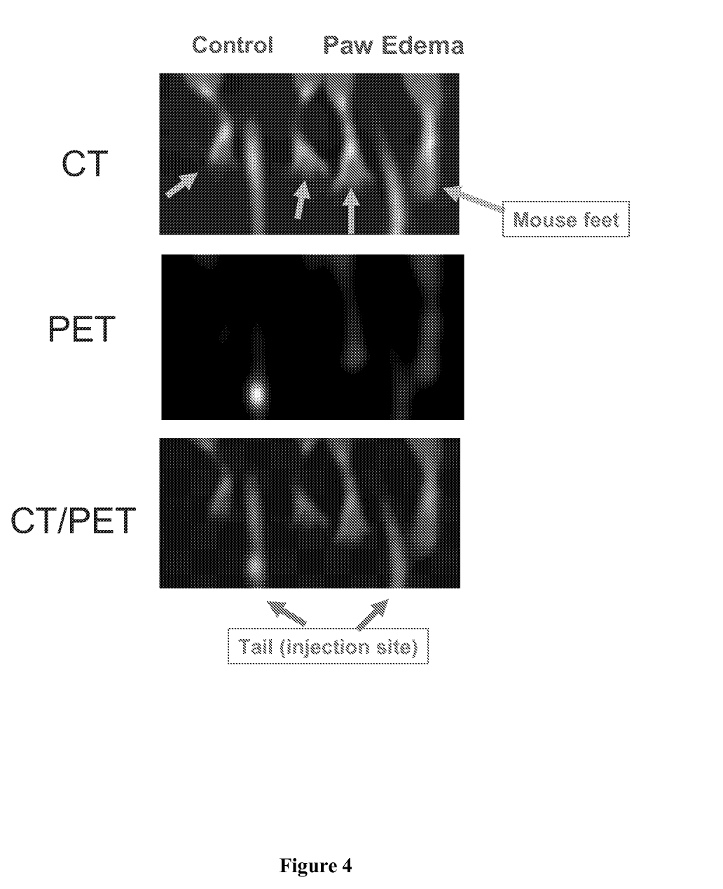

In vivo biodistribution studies were performed to determine whether [¹⁸F]M508F can be used to image CXCR4-positive inflammation in vivo. [¹⁸F]M508F-PET imaging of CXCR4-positive paw edema in mice exhibited significant uptake of our CXCR4-PET tracer at 30 mins post-injection (FIG. 4).

### Example 6

### MicroPET/CT Imaging Study of [¹⁸F]M508F in a Lung Metastasis of SCCHN Animal Model

MicroPET/CT scan images of the lungs were performed to determine whether [¹⁸F]M508F can be used to image CXCR4-positive lung metastases in vivo. These images were obtained 60 minutes post-injection of [¹⁸F]M508F. These images exhibit significant uptake of our CXCR4-PET tracer by metastatic SCCHN tumors at 60 mins post-injection.

### Example 7

### MicroPET/CT Imaging Study of [¹⁸F]M508F in a Lung Inflammation Induced by the Strong-Dose Radiation Therapy

MicroPET/CT scan images of the lungs were performed to determine whether [¹⁸F]M508F can be used to image lung inflammation induced by the strong-dose radiation therapy in vivo. These images were obtained 60 minutes post-injection of [¹⁸F]M508F. These images exhibit significant uptake of our CXCR4-PET tracer by CXCR4-positive immune cells that got recruited by the inflammation process at 60 mins post-injection.

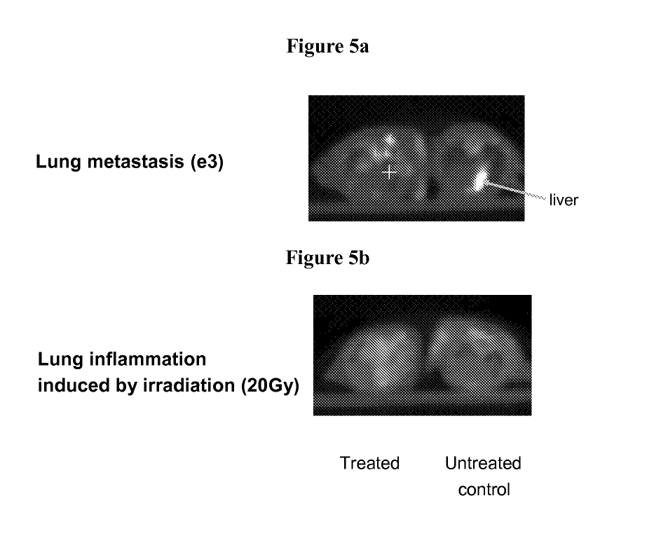

FIG. 5b shows PET/CT scan images of the lungs of control and mouse with lung inflammation due to 20 Gy—irradiation. These images were obtained 60 minutes post-injection of [¹⁸F]M508F.

1. An imaging composition comprising a labeled CXCR4 antagonist.

2. The imaging composition of claim 1, wherein the labeled CXCR4 antagonist comprises an element that produces a radioactive emission.

3. The imaging composition of claim 1, wherein the labeled CXCR4 antagonist comprises a radioisotope selected from the group consisting of ¹⁸F, ⁷⁶Br, ¹²³I, ¹²⁴I, ¹²⁵I, and ¹³¹I.

4. A compound of Formula I, salt, ester or prodrug thereof: 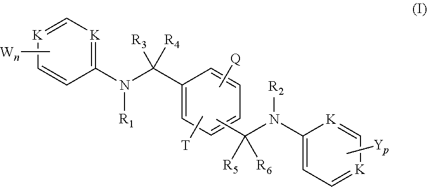
wherein:
each K is independently N, CH or CX where each X is independently selected from straight chain, branched or cyclic alkyl, heteroalkyl, haloalkyl, aralkyl, aryl, heteroaryl, F, Cl, Br, I, NH₂, NHR, NR₂, SR, S₂R, S—NHR, S₂—NHR, S—NRR′, S₂—NRR′, NHacyl, N(acyl)₂, CO₂H, CO₂R, CONRR′, or CN;
each Q, T, W and Y are each independently H, R, acyl, F, Cl, Br, I, OH, OR, NH₂, NHR, NR₂, SR, S₂R, S—NHR, S₂—NHR, S—NRR′, S₂—NRR′, NHacyl, N(acyl)₂, CO₂H, CO₂R, CONRR′ or CN, where R and R′ are independently selected from straight chain, branched or cyclic alkyl, alkenyl, alkynyl, aminoalkyl, heteroalkyl, haloalkyl, aralkyl, aryl, heteroarylalkyl and heterocyclylalkyl;
wherein at least one of Q, T, W and Y is a radioisotope selected from the group consisting of ¹⁸F, ⁷⁶Br, ¹²³I, ¹²⁴I, ¹²⁵I, and ¹³¹I;
n is 0, 1, 2 or 3;
p is 0, 1, 2 or 3; and
R¹, R², R³, R⁴, R⁵ and R⁶ are independently selected from H, straight chain, branched or cyclic alkyl, alkenyl, alkynyl, aralkyl, aryl heteroaryl, acyl and imidoyl groups.

5. The compound of claim 4, wherein at least one of Q, T, W and Y is ¹⁸F.

6. The compound of claim 4, wherein R¹ and R² are each H or alkyl.

7. The compound of claim 4, wherein R³, R⁴, R⁵ and R⁶ are each H or alkyl.

8. A pharmaceutical composition comprising a compound of claim 4 or pharmaceutically acceptable salt thereof and a pharmaceutically acceptable carrier.

9. A method of producing a radiographic image comprising
a) administering a pharmaceutical composition of claim 2 to a host;
b) detecting radioactive emissions at a location of the host; and
c) creating a radiographic image with an imaging apparatus.

10. A method of detecting cancer comprising;
a) administering a pharmaceutical composition of claim 2 to a host;
b) producing a radiographic image; and
c) correlating radiographic image intensity in the host to the existence of a cancer.

11. A method of determining the metastatic potential of a tumor comprising;
a) administering a pharmaceutical composition of claim 2 to a host diagnosed with a tumor;
b) producing a radiographic image; and
c) correlating radiographic image intensity in the host to the existence of a metastatic tumor.

12. The method of claim 9, wherein producing the radiographic image is by positron emission tomography.

13. A compound N⁴-(4-((2-¹⁸-fluoro,5-fluoropyrimidin-4-ylamino)methyl)benzyl)-N²-(2-morpholinoethyl)pyrimidine-2,4-diamine or salt thereof.

14. A compound of the following formula: 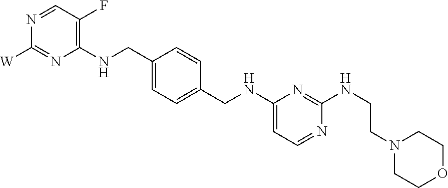
or salt thereof wherein W is F, Br or I.

15. A method of producing N⁴-(4-((2-¹⁸-fluoro,5-fluoropyrimidin-4-ylamino)methyl)benzyl)-N²-(2-morpholinoethyl)pyrimidine-2,4-diamine comprising mixing N⁴-(4-((2-halogen,5-fluoropyrimidin-4-ylamino)methyl)benzyl)-N²-(2-morpholinoethyl)pyrimidine-2,4-diamine and a composition comprising ¹⁸F under conditions such that N⁴-(4-((2-¹⁸-fluoro,5-fluoropyrimidin-4-ylamino)methyl)benzyl)-N²-(2-morpholinoethyl)pyrimidine-2,4-diamine is formed.
